(19) United States
(12) Patent Application Publication
(10) Pub. No.: US 2009/0192297 A1
(43) Pub. Date: Jul. 30, 2009

(54) CARBON MEMBRANE HAVING BIOLOGICAL MOLECULE IMMOBILIZED THEREON

(75) Inventors: Youichi Yoshida, Ube-shi (JP); Shinichiro Sadaike, Ube-shi (JP); Shyusei Ohya, Ichihara-shi (JP); Kikuo Ataka, Ube-shi (JP)

Correspondence Address: KNOBBE MARTENS OLSON & BEAR LLP, 2040 MAIN STREET, FOURTEENTH FLOOR, IRVINE, CA, 92614, US

(73) Assignee: Ube Industries, Ltd., Yamaguchi (JP)

(21) Appl. No.: 12/278,066

(22) PCT Filed: Feb. 2, 2007
(86) PCT No.: PCT/JP2007/051813

§ 371 Date: Oct. 10, 2008

(30) Foreign Application Priority Data
Feb. 2, 2006 (JP) 2006-025504

(51) Int. Cl. C07K 17/14

(52) U.S. Cl. 530/402

(57) ABSTRACT

Disclosed is a biological molecule-immobilized carbon membrane which comprises a porous carbon membrane and a biological molecule (e.g., an enzyme) immobilized on the carbon membrane, wherein the porous carbon membrane has three-dimensional cancellous pores through which fluid can permeate. The carbon membrane can have a large amount of a biological molecule (e.g., an enzyme) immobilized thereon and can also have a higher level of enzymatic activity or the like compared to a conventional one. Therefore, the carbon membrane is useful as an electrode for a bio-sensor or a bio-fuel cell.

### FIELD OF THE INVENTION

The present invention relates to a porous carbon membrane, in particular a carbon membrane on which a biological molecule is immobilized, and the applications of that carbon membrane in the use of electrodes, battery materials, sensors, semiconductor devices and so on.

### BACKGROUND ART

Researches on immobilizing biological molecules on electrodes such as carbon and the application using them as sensors and power-generating elements are in progress (Patent references 1 and 2, and Non-patent references 1 to 4). How many amounts of the biological molecules can be immobilized on an electrode is a very important factor to determine the sensitivity of a sensor and the output power of a power-generating element.

For example, JP A 2005-83,873 (Patent reference 1) describes a bio-sensor in which a biologically originating molecule such as an enzyme is immobilized on the surface of a carbon material by covalently bonding through a molecule such as cyanuric chloride or absorption. The electrode carbon material is obtained by mixing chloridized vinyl chloride resin etc. and graphite particles, followed by calcination. Since, however, this carbon material is not a fluid-permeable or porous material, the biologically originating molecule is merely immobilized on the plate-face of electrode.

JP A 2005-60,166 (Patent reference 2) also describes a carbon-coated electrode in which the porous surface of a substrate such as silicon having a vertical column-shape pore is coated with carbon. Although this electrode has an increased carbon surface area, its production is troublesome because it is a composite material. Its application is also limited because it has no permeability for gas or fluid and there is no connection between adjacent pores.

A mediator for mediating a redox reaction is generally required to make a biological molecule electrode function. In a previously known method, an enzyme and mediator were confined in a three-dimensional gel formed by adding an epoxy resin to a component primarily containing an amino group to immobilize them on an electrode. In the non-patent reference 3, an enzyme and mediator are immobilized on a carbon fiber with a diameter of 7 μm by the above three-dimensional gel method to construct a bio-fuel cell, whereby an output power of 137 μW/cm² is obtained. The non-patent reference 5 also describes a process for co-immobilizing an enzyme-mediator by the layer-by-layer stacking method (or layer-by-layer adsorption method) where a solid substrate is alternately immersed into positive and negative polymeric electrolyte aqueous solutions, respectively. In terms of the comparison of the three-dimensional gel and layer-by-layer stacking methods, non-patent reference 6 has compared both the three-dimensional gel method and layer-by-layer stacking method for immobilizing a mediator and enzyme on a glassy-carbon electrode, and has reported that the three-dimensional gel method is superior.

Patent reference 1: JP A 2005-83,873

Patent reference 2: JP A 2005-60,166

Non-patent reference 1: Analytical Letters, 32(2), 299-316 (1999)

Non-patent reference 2: Bioelectrochemistry 55 (2002)29-32

Non-patent reference 3: Journal of American Chemical Society 2001, 123, 8630-8631

Non-patent reference 4: Chemical Review 2004, 104, 4867-4886

Non-patent reference 5: Analytical Chemistry, vol. 78, 399, 2006

Non-patent reference 6: The 9th Biological Catalytic Chemistry Symposium (Jan. 27, 2006), Poster Presentation, Kanoh et al., page 10

### SUMMARY OF THE INVENTION

As above, trials have been previously made for immobilizing biological molecule or biological molecule and mediator. However, they are insufficient in the performance and further improvements has been demanded.

An object of the present invention is to provide a biological molecule-immobilized carbon membrane having large amounts of the immobilized biological molecule and a higher level of functionality of the biological molecule than the previous level. Another object of one aspect of the present invention is to provide a sensor and bio-fuel cell having an excellent enzyme activity and electric response. An object of another aspect of the present invention is to provide a novel functional membrane suitable for immobilizing biological molecules).

The present invention relates to the following items.

1. A biological molecule-immobilized carbon membrane, wherein biological molecule is immobilized onto a porous carbon membrane having fluid-permeable three-dimensional cancellous pore.
2. A biological molecule-immobilized carbon membrane according to above item 1, wherein the porous carbon membrane has an air permeability of 10 to 2,000 sec/100 cc, and a specific surface area of 1 to 1,000 m²/g.
3. A biological molecule-immobilized carbon membrane according to above item 1 or 2, wherein an electrostatic interaction of the porous carbon membrane surface and the biological molecule causes the immobilization of the biological molecule.
4. A biological molecule-immobilized carbon membrane according to above item 3, wherein an anion group is introduced onto the surface of the porous carbon membrane by an oxidation treatment, and the electrostatic interaction of this surface anion group and a positive charge in the biological molecule causes the immobilization of the biological molecule.
5. A biological molecule-immobilized carbon membrane according to above item 3, wherein a compound having a cation group is introduced onto the surface of the porous carbon membrane after an oxidation treatment, and the electrostatic interaction of this surface cation group and a negative charge in the biological molecule causes the immobilization of the biological molecule.
6. A biological molecule-immobilized carbon membrane according to above item 1 or 2, wherein a covalent bond between a surface of the porous carbon membrane and the biological molecule causes the immobilization of the biological molecule.
7. A biological molecule-immobilized carbon membrane according to above item 1 or 2, wherein a physical interaction of the porous carbon membrane surface and the biological molecule causes the immobilization of the biological molecule.
8. A biological molecule-immobilized carbon membrane according to above item 1 or 2, comprising a first polymeric electrolyte having a charge opposite to a charge of the biological molecule, and forming an ion complex by an electrostatic interaction with the biological molecule.
9. A biological molecule-immobilized carbon membrane according to above item 8, wherein the biological molecule and the first polymeric electrolyte are alternately stacked to form the ion complex.
10. A biological molecule-immobilized carbon membrane according to above item 8 or 9, further comprising a second polymeric electrolyte having a charge same to a charge of the biological molecule, and forming the ion complex with the first polymeric electrolyte in a manner where the biological molecule and the second polymeric electrolyte are mixed.
11. A biological molecule-immobilized carbon membrane according to any one of above items 8 to 10, wherein the anion group is introduced onto the surface of the porous carbon membrane before introducing the biological molecule.
12. A biological molecule-immobilized carbon membrane according to any one of above items 8 to 10, wherein the anion group is introduced onto the surface of the porous carbon membrane before introducing the biological molecule, followed by a treatment with an organic solvent solution of the compound having the cation group.
13. A biological molecule-immobilized carbon membrane according to any one of above items 1 to 12, wherein the biological molecule is a protein or a nucleotide.
14. A sensor comprising the biological molecule-immobilized carbon membrane according to any one of above items 1 to 12 as an electrode.
15. A biofuel cell comprising the biological molecule-immobilized carbon membrane according to any one of above items 1 to 12 as an electrode.
16. A process for producing a biological molecule-immobilized carbon membrane, comprising steps of:

providing a porous carbon membrane having a three-dimensional cancellous pore, an air permeability from 10 to 2,000 sec/100 cc and a specific surface area from 1 to 1,000 m²/g,

oxidation-treating the porous carbon membrane, and

immersing the porous carbon membrane after oxidation-treated in a solution containing the biological molecule to immobilize the biological molecule onto the porous carbon membrane.

17. A process for producing a biological molecule-immobilized carbon membrane, comprising steps of:

providing a porous carbon membrane having a three-dimensional cancerous pore, an air permeability from 10 to 2,000 sec/100 cc, and a specific surface area from 1 to 1,000 m²/g,

oxidation-treating the porous carbon membrane,

introducing a cation group onto a surface of the porous carbon membrane after oxidation-treated, and

immersing the porous carbon membrane after the cation group has been introduced in a solution containing the biological molecule to immobilize the biological molecule onto the porous carbon membrane.

18. A process for producing a biological molecule-immobilized carbon membrane, comprising steps of:

providing a porous carbon membrane having a three-dimensional cancellous pore, an air permeability from 10 to 2,000 sec/100 cc, and a specific surface area from 1 to 1,000 m²/g,

oxidation-treating the porous carbon membrane, and

immobilizing the biological molecule onto the porous carbon membrane through a covalent bond.

19. A process for producing a biological molecule-immobilized carbon membrane, comprising steps of:

providing a porous carbon membrane having a three-dimensional cancerous pore, an air permeability from 10 to 2,000 sec/100 cc, and a specific surface area from 1 to 1,000 m²/g, and

bringing a mixture containing the biological molecule and a crosslinkable compound into contact with the porous carbon membrane to immobilize the biological molecule onto the porous carbon membrane.

20. A functional carbon membrane, wherein is oxidized a surface of a porous carbon membrane having a fluid-permeable three-dimensional cancellous pore, followed by introducing a compound having a cation group.
21. A functional carbon membrane according to above item 20, wherein an air permeability of the porous carbon membrane is 10 to 2,000 sec/100 cc, and a specific surface area is 1 to 1,000 m²/g.
22. A biological molecule-immobilized carbon membrane according to above item 13, wherein the biological molecule is selected from the group consisting of glucose dehydrogenase, glucose oxidase, bilirubin oxidase, diaphorase, alcohol dehydrogenase, avidin and biotin.
23. A process for producing a biological molecule-immobilized carbon membrane, comprising the steps of:

providing a porous carbon membrane having three-dimensional cancellous pore, an air permeability from 10 to 2,000 sec/100 cc, and a specific surface area from 1 to 1,000 m²/g,

providing a solution (a) and a solution (b), wherein the solution (a) contains one or more polymeric electrolytes with a positive charge and the solution (b) contains one or more polymeric electrolytes with a negative charge, and wherein at least one of the polymeric electrolyte with the positive charge and the polymeric electrolyte with the negative charge is the biological molecule, and

alternate stacking by at least each one time conducting alternately sub-steps of:

(a) immersing the porous carbon membrane in the solution (a) and

(b) immersing the porous carbon membrane in the solution (b).

24. A production process according to above item 23, comprising the step of oxidation-treating the porous carbon membrane before the alternate stacking, and

wherein the sub-step (a) is conducted in first during the alternate stacking subsequently.

25. A production process according to above item 23,

wherein the production process comprising, prior to the step of alternate stacking, steps of oxidation-treating the porous carbon membrane, and introducing a cation group onto a surface of the porous carbon membrane after oxidation-treated; and

wherein the subsequent step of alternate stacking starts from the sub-step (b).

26. A production process according to any one of above items 23 to 25, wherein one of either the solution (a) or the solution (b) contains the biological molecule, and the alternative other contains a mediator.
27. A production process according to any one of above items 23 to 26, wherein one of either the solution (a) or the solution (b) contains both the biological molecule and the mediator.

### EFFECT OF THE INVENTION

According to the present invention, there is provided a biological molecule-immobilized carbon membrane having large amounts of the immobilized biological molecule and a higher level of functionality of the biological molecule than the previous level. In the biological molecule-immobilized carbon membrane of the present invention, the biological molecules are usually immobilized in such a state that they are dispersed over the entire membrane, which enables the membrane to have an excellent biological molecule activity represented by an enzyme activity. When, therefore, the biological molecule-immobilized carbon membrane of the present invention is used for a sensor electrode, a large electric response is obtained, which enables high sensitivity, detection with a low concentration, and miniaturization. When, furthermore, the biological molecule-immobilized carbon membrane of the present invention is used for the electrode of a bio-fuel cell, it is advantageous for a practical application due to its large output power.

### BRIEF DESCRIPTION OF THE DRAWINGS

FIG. 1 is a scanning electron microscope image of the surface of the porous carbon membrane produced in the referential example 2.

FIG. 2 is a scanning electron microscope image of the cross section of the porous carbon membrane produced in the referential example 2.

FIG. 3 is an XPS spectrum of the porous carbon membrane (a) before the PEI treatment and (b) after the PEI treatment.

FIG. 4 is a scanning electron microscope image of the surface of the porous carbon membrane before the immobilization with ferritin.

FIG. 5 is a scanning electron microscope image of the surface of the porous carbon membrane after the immobilization with ferritin.

FIG. 6 is a scanning electron microscope image of the surface of the porous carbon membrane after the immobilization with ferritin and further calcination.

FIG. 7 is a graph showing (a) the micropore distribution and (b) the surface area for the porous carbon membranes of “untreated”, “after treated with nitric acid”, “after the PEI treatment”, and “after the PEI treatment-GOX immobilization”.

FIG. 8 is a graph showing an electric current output of the GDH immobilized electrode of the present invention and the GDH immobilized electrode of the comparative example, each in a range of a low glucose concentration.

FIG. 9 is a result of EPMA (Electron-ray Probe Micro Analyzer) analysis over the cross section of the membrane after the ferritin immobilization. In the line profile, the above and below is a distance in the membrane cross section, and the right side represents a Fe concentration. In the image, the darker black represents the higher concentration.

FIG. 10 is a graph showing the responding electric current when the number of the stacked layers is changed in the layer-by-layer stacking method.

FIG. 11 is a graph showing an electric current output of the electrodes in a range of a low concentration of glucose. One of the electrodes is formed by applying the layer-by-layer stacking method to the porous carbon membrane and another is formed by applying the same method to a carbon paper.

FIG. 12 is a graph comparing the molecular weight of polyacrylic acids when the layer-by-layer stacking method is applied.

FIG. 13A is a figure showing an example of the sensor structure for the flow injection analysis.

FIG. 13B shows a cross section of the sensor structure shown in the FIG. 13A.

FIG. 14 is a graph showing the relationship of the glucose concentration and output electric current by the flow injection analysis.

FIG. 15A is a figure showing an example for the structure of the chip-type bio-fuel cell.

FIG. 15B is a cross section of the structure of the chip-type bio-fuel cell shown in the FIG. 15A.

FIG. 15C is a figure showing a structural example for the single cell used for the chip-type bio-fuel cell shown in the FIG. 15A.

FIG. 16 is a figure showing an example for the structure of the polymeric electrolyte membrane-type bio-fuel cell.

FIG. 17 is a result of the EPMA analysis of the cross section of the membrane after the immobilization with ferritin produced according to the example 11.

FIG. 18 is a result of the EPMA analysis of the cross section of the membrane after the immobilization with ferritin produced according to the example 17.

FIG. 19 is a scanning electron microscope image of the cross section of the membrane after the immobilization with ferritin produced according to the example 17.

### DETAILED DESCRIPTION OF THE INVENTION

### Porous Carbon Membrane Having Three-Dimensional Cancellous Pores

The porous carbon membrane having three-dimensional cancellous pores used in the present invention is capable of ventilating gas and fluid because the pores of the membrane are mutually connected (i.e. pores are communicated with each other). The level of mutual connection of the pores is expressed by the air permeability measured in accordance with JIS P8117 (as later described in detail), and is preferably 1 to 2,000 sec/100 cc, particularly preferably 10 to 2,000 sec/100 cc. The BET specific surface area is usually 1 to 1,000 m²/g, preferably 3 to 200 m²/g, particularly preferably 5 to 30 m²/g. Also, the vacancy ratio is preferably 20 to 80%, particularly preferably 30 to 60%. The vacancy ratio can be calculated by the weight method after determining the true density. The mean pore diameter is preferably 10 to 1,000 nm, particularly 50 to 500 nm according to the measurement of the bubble point method (ASTM F316, JISK 3832) (as later described in detail).

The carbon content of the porous carbon membrane may be arbitrarily varied to meet the purpose of use; however, it is preferably 80 atomic % or more, in a particular application preferably 95 atomic % or more. Since the present invention is particularly used for the sensor electrode or the bio-fuel cell electrode, those having high carbon content and electric conductivity are preferable. Consequently, supplementary conducting agents etc. are not needed because an electrode can be constructed by utilizing the electric conductivity of the membrane substrate.

The form (shape etc) of the porous carbon membrane is not especially limited as long as it has property as described above. In some applications, the membrane may possess a form of network that is formed by entwined fibrous carbon. In general, preferred is a porous membrane in which frothy voids are mutually connected (i.e. communicating one another). The latter membrane may be obtained by carbonizing the porous membrane made from a high heat resistance resin of polyimide-bases, cellulose-bases, furfural resin-bases, phenol resin-bases and the like, as described in, for example, JP A 2000-335909 and JP A 2003-128409. Particularly preferable porous carbon membrane is that in which a polyimide precursor is precipitated from a polyimide precursor solution such as polyamic acid to prepare porous form, followed by polyimidizing and carbonizing.

<Surface Treatment of the Porous Carbon Membrane>

The porous carbon membrane on which the biological molecule(s) is immobilized in the present invention, may include three types; that is, on the porous carbon membrane surface, (1) an anion group is introduced, (2) a cation group is introduced, and (3) untreated or hydrophobic state. Since the surface of the carbonized membrane is usually hydrophobic, the immobilization of the biological molecule is usually carried out without any treatments when the hydrophobic surface described previously in (3) is sought.

Here, the anion group means a group bearing negative charge (also including the case it already becomes negative charge) due to the ambient pH when the biological molecule is immobilized, for example it includes those an acid group such as —COOH (or —COO⁻), —SO₃H (or —SO₃⁻) and —PO₄H₂ (or —PO₄H⁻) is introduced onto the surface. In this case, it may be introduced directly onto the carbon surface, or the above-described anion group may be introduced as a part of a molecule. For the anion group, —COOH (or —COO⁻) is particularly preferable.

Although the introduction of the anion group may be conducted by a suitable treatment for the group intended to be introduced, a method of oxidation-treatment of the surface is one of the concise methods, and by which COOH group is thought to be introduced. It preferably includes a treatment with a nitric acid aqueous solution (nitric acid oxidation), hydrogen peroxide oxidation, a high temperature treatment in the presence of steam in the air, and an oxygen plasma treatment, more preferably a treatment with a nitric acid aqueous solution. By selecting a condition, the amount of the anion group to be introduced may be adjusted. In the case of the nitric acid oxidation, the amount of carboxylic acid on the surface may be varied by selecting a nitric acid concentration, reaction time, and reaction temperature. The nitric acid concentration is preferably 5 to 69%, particularly preferably 10 to 60%. The reaction temperature is preferably 10° C. to 120° C., particularly preferably 50° C. to 120° C. The reaction time is preferably 0.5 to 60 hours, particularly preferably 1 to 30 hours. The anion group may also be introduced by the reaction with the carboxylic acid group which has been introduced onto the surface by the oxidation treatment.

Next, the cation group means a group bearing positive charge (also including the case it already becomes positive charge) due to the ambient pH when the biological molecule is immobilized, for example it includes primary amine group {—NH₂}, secondary amine group {(−)₂NH}, tertiary amine group {(−)₃N}, quaternary amine group {(−)₄N+}, and imidazole. These groups may be introduced directly onto the carbon surface, or these cation groups may be introduced as a part of a molecule. In particular, the cation group is preferably introduced as a part of a molecule.

The introduction of the cation group may be conducted by a suitable treatment for the group intended to be introduced. For example, it includes an oxygen plasma treatment in the presence of ammonia; more preferably the introduction of the cation group in which the surface is first oxidation-treated to introduce a carboxylic acid group and increase the amount of the functional group because there is few functional groups on the surface of the untreated carbon membrane, followed by conducting various reactions toward the carboxylic acid group.

When, in particular, a compound molecule possessing a cation group is introduced, the introduced compound molecule should possess, together with the cation group, a reaction group capable of reacting with a functional group such as COOH on the carbon membrane surface (this reaction group may also be a cation group). Also preferred is a method in which COOH on the porous carbon membrane is treated with thionyl chloride and the like to form acid chloride so as to increase its reactivity, then cation group is introduced. A group capable of reacting with the —COOH or —COCl group on the porous carbon membrane surface includes primary amine group {—NH₂}, secondary amine group {(−)₂NH}, and hydroxyl group {—OH}.

Taking polyethylenimine for instance as a compound possessing a cation group, it may be introduced onto the surface of the porous carbon membrane as follows.

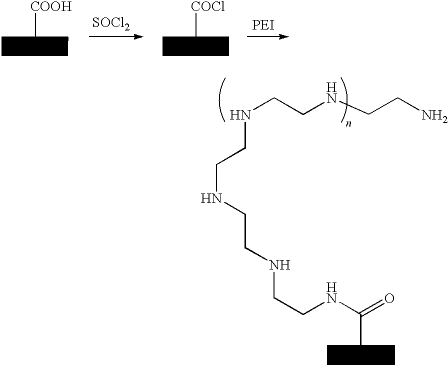

In this polyethylenimine compound, the repeating number of the ethylenimine unit may be arbitrarily varied to meet its properties required.

Other than the polyethylenimine compound, compounds possessing a functional group such as primary or secondary amine group capable of reacting with the COCl on the carbon membrane surface and the cation group such as primary to tertiary amine group may be introduced onto the porous carbon membrane surface in a similar manner to the above-described scheme. For example, they include polymer or oligomer of basic amino acid such as lysine, arginine, and ornithine, and other polymers or oligomers containing these basic amino acids. Although the polyethylenimine is bonded with the carbon membrane surface at a single site in the above-described scheme, they may be bonded at multiple sites.

Although the figure shows the covalent bond with the COOH of the carbon surface in the above-described scheme, a compound possessing the cation group (the above-described polyethylenimine etc.) may be also introduced by the electrostatic bond with COO⁻ from the electrolytic dissociation of the surface COOH group.

To introduce the compound possessing these cation groups, the above-described compound as-is, if it is fluid, may be brought into contact with the carbon membrane, or a solution in a solvent such as water and/or an organic solvent may be brought into contact with the carbon membrane when it is fluid or solid. When a solvent is used, it preferably has a high affinity with the carbon membrane and also has a low viscosity. When the compound possessing the cation group is introduced by an electrostatic bond, for example, alcohols such as methanol and ethanol may be used.

The membrane in which the cation group is introduced onto the surface of the porous carbon membrane possessing three-dimensional cancellous pores by this way has not existed previously and it is a novel functional carbon membrane. In addition to the immobilization of the biological molecule(s), it is useful for various reactions utilizing the surface cation and various applications such as a carrier for example, to support a metal fine particle. In particular, it is useful for an application that utilizes an electric conducting property simultaneously.

<Immobilization of the Biological Molecule>

The biological molecule to be immobilized onto the porous carbon membrane in the present invention includes protein such as enzyme, antigen and antibody; nucleic acid such as oligonucleotide, polynucleotide and gene; lipid; and carbohydrate. Protein such as enzyme, antigen and antibody is particularly preferable.

A method for immobilizing the biological molecule(s) onto the porous carbon membrane in the present invention includes (1) a method utilizing the electrostatic interaction of the charge of the porous carbon membrane surface and the charge of the biological molecule, (2) a method covalently binding between the surface of the porous carbon membrane and the biological molecule, optionally via a molecule cluster, and (3) a method utilizing the physical interaction of the porous carbon membrane surface and the biological molecule, optionally with the aid of the physical interaction of other compound.

Among three, (1) a method utilizing the electrostatic interaction of the charge of the porous carbon membrane surface and the charge of the biological molecule is the most preferable. Many biological molecules generally possess group(s) capable of electrolytic dissociation, and bear positive charge or negative charge depending on pH of aqueous solution. Protein such as enzyme, antigen and antibody bears positive charge (cation) at a pH below its isoelectric point, and bears negative charge (anion) at a pH above its isoelectric point.

On the other hand, the porous carbon membrane used for this immobilization method, in which an anion group or a cation group is introduced onto its surface, electrolytically dissociates at an appropriate pH in an aqueous medium. Hence, the biological molecule is electrostatically immobilized on the membrane surface by bringing the porous carbon membrane into contact with a solution of the biological molecule under an appropriate pH. In particular, the present invention can provide both of porous carbon membranes, one of which is that the anion group is introduced onto the surface and another of which is that the cation group is introduced onto the surface. Therefore, the suitable porous carbon membrane surface-treated can be selected by considering a pH for immobilization and a pH for using the biological molecule-immobilized membrane. This allows broad range of biological molecule that can be immobilized. Since, furthermore, the electrostatic interaction scarcely changes the biological molecule and decreases the biological molecule activity, the range of the biological molecule applicable is also broad from this point. Since it is also based on the electrostatic interaction of the anion group or the cation group introduced onto the surface, the biological molecule(s) can be readily immobilized uniformly with excellent dispersibility. Since, in addition, the biological molecule(s) is located close to the carbon surface, the interaction with the carbon is large and it is considerably advantageous for giving and receiving electron. It is preferable as the functional electrode such as the sensor electrode and the bio-fuel cell electrode.

As a specific example for immobilization, the later-described ferritin may be immobilized onto the porous carbon membrane oxidized with nitric acid at a pH less than the isoelectric point of 4.79, for example a pH around 4.3. Since glucose oxidase bears negative charge under near-neutral condition, it may be immobilized at a pH around 7 onto the porous carbon membrane oxidized with nitric acid, followed by introducing polyethylenimine onto the surface and introducing the cation group onto the surface. Since PQQ-dependent glucose dehydrogenase bears positive charge under near neutral condition, it may be immobilized at a pH around 7 onto the porous carbon membrane oxidized with nitric acid.

The biological molecule immobilizable onto the porous carbon membrane surface by this method includes enzyme such as glucose dehydrogenase (NAD-dependent and PQQ-dependent), glucose oxidase, bilirubin oxidase, diaphorase, and alcohol dehydrogenase; and protein such as avidin and biodin.

Next, is explained (2) a method covalently binding between the surface of the porous carbon membrane and the biological molecule, optionally via a molecule cluster. In this method, a functional group in the biological molecule is involved with covalent bond to immobilize it on the surface of the porous carbon membrane.

As a specific method, it is possible to apply a known method such as the cyanuric chloride method, the γ-aminopropyltriethoxysilane-glutaraldehyde method, the carbodiimide dehydration condensation method, and the thionyl chloride method as described in JP A 2005-83873. For example, in the cyanuric chloride method, after the porous carbon membrane is optionally subjected to treatment such as the nitric acid oxidation, cyanuric chloride is contacted and followed by contacting with protein etc. to cause the covalent bond between the cyanuric compound and the amino group of the protein. It is also possible to utilize a reaction with the sugar chain of the protein. In the γ-aminopropyltriethoxysilane-glutaraldehyde method, the porous carbon membrane is also treated with γ-aminopropyltriethoxysilane to introduce the (—O—)₃Si—(CH₂)₃—NH₂ group onto the surface, followed by forming the Schiff's base with one aldehyde group of glutaraldehyde and further reacting the other aldehyde group with the amino group of the protein to cause the covalent bond by forming the Schiff's base. In the carbodiimide dehydration condensation method and the thionyl chloride method, amide bond is finally produced by the reaction with the amino group of the protein. It is also possible to form ester bond with OH group of the biological molecule.

In the method to immobilize the biological molecule by the covalent bond, the biological molecule is required to possess a functional group involved in the reaction. The functional group in the biological molecule to be utilized for the immobilization includes primary amino group, secondary amino group, and OH group as above described for example. In the case of protein having lysine residue, it is possible to utilize the NH₂ thereof. In addition, in use of the immobilization by the covalent bond, the biological molecule are selected from those of which the functions (enzymatic activity and antigen-antibody reaction) are not considerably reduced after the reaction. From this point in comparison with the electrostatic bond, the restriction to the biological molecule to be immobilized is increased. In, nevertheless, this method, the biological molecule can also be readily immobilized uniformly with excellent dispersibility, and the interaction with the carbon is large and it is considerably advantageous for giving and receiving electron because the biological molecule is located close to the carbon surface. It is preferable as the functional electrode such as the sensor electrode and the bio-fuel cell electrode.

The biological molecule immobilizable onto the porous carbon membrane surface by this method includes enzyme such as glucose dehydrogenase (NAD-dependent and PQQ-dependent), glucose oxidase, bilirubin oxidase, diaphorase, and alcohol dehydrogenase; and protein such as avidin and biodin.

(3) The immobilization method by the physical interaction of the porous carbon membrane surface and the biological molecule is a method utilizing physical adsorption such as hydrophobic bond, in which the biological molecules) does not have chemical bond with the porous carbon membrane surface. Even in the physical interaction, in particular if the biological molecules are cross-linked (hereafter, also referred to as the cross-linking method), dropout of molecules decreases and the immobilization also becomes stronger. For example, it is preferable to cross-link the biological molecules by forming the Schiff's base between glutaraldehyde and the amino group of the biological molecule. Since the function (enzymatic activity and antigen-antibody reaction) of the biological molecule may be considerably reduced if all the amino groups react in this reaction, it is also preferable to mix other proteins such as bovine serum albumin, polylysine, and polyethylenimine.

The biological molecule immobilizable onto the porous carbon membrane surface by this method includes enzyme such as glucose dehydrogenase (NAD-dependent and PQQ-dependent), glucose oxidase, bilirubin oxidase, diaphorase, and alcohol dehydrogenase; and protein such as avidin and biodin.

To contact solution containing the biological molecule in the pores of the porous carbon membrane in a practical operation for the immobilization method above descried (1) to (3), the porous carbon membrane is immersed in the biological molecule solution, and the pores inside are once deaerated under a reduced pressure, followed by recovering to an ordinary pressure to allow the solution impregnates with even into the fine pores. This enables the immobilization of the biological molecule even inside the fine pores.

The biological molecule-immobilized carbon membrane produced by this in the present invention holds the three-dimensional cancellous structure even after the immobilization of the biological molecule, and it has the air permeability of 1 to 2,000 sec/100 cc, particularly preferably 10 to 2,000 sec/100 cc. This is because the present invention can immobilize the biological molecule onto the pore surfaces with less occurrence of its aggregation by selecting appropriate conditions.

<Immobilization of the Biological Molecule by the Layer-By-Layer Stacking Method>

The Layer-by-Layer Stacking method (Layer-by-Layer Adsorption (LBL) method) is a method in which a substrate is alternately immersed in solutions of positive and negative polymeric electrolytes respectively to prepare a polyion complex insoluble in water in step-by-step manner. In the electrode application of the sensor and bio-fuel cell, it is important to immobilize a large amount of the biological molecule having an utilizable form on the electrode. The present inventors have discovered that the immobilization amount of the biological molecule can be increased without obturation of the porous carbon membrane pores and with the state maintaining its air permeability by applying the layer-by-layer stacking method.

In the present invention, the porous carbon membrane is subjected to the sub-step (a): the sub-step of immersing in the solution (a) containing polymeric electrolyte with positive charge and the sub-step (b): the sub-step of immersing in the solution (b) containing polymeric electrolyte with negative charge, each one time or more. During this step, it is possible to immobilize the biological molecule onto the porous carbon membrane by using the biological molecule as at least one of the polymeric electrolyte with positive charge contained in the solution (a) and the polymeric electrolyte with negative charge contained in the solution (b).

Here, the polymeric electrolyte may be available as long as it dissolves in solution (usually aqueous solution) and bears electrical charge, and the examples thereof include naturally-occurring high-molecular compounds and synthetic high-molecular compounds such as polymers. Although its molecular weight is not particularly restricted, those having 1,000 or more in a mass-average molecular weight particularly 5,000 or more are preferable.

The polymeric electrolyte with positive charge contained in the solution (a) and/or the polymeric electrolyte with negative charge contained in the solution (b) may comprise a single kind or multiple kinds.

By this step, the biological molecule and the first polymeric electrolyte having the charge opposite to the charge of the biological molecule, forms ion complex by the electrostatic interaction, and they are immobilized on the porous carbon membrane. When the solution (a) contains the biological molecule, the first polymeric electrolyte is one of the polymeric electrolytes contained in the solution (b). When the solution (b) contains the biological molecule, the first polymeric electrolyte is one of the polymeric electrolytes contained in the solution (a).

When the solution containing the biological molecule further contains the second polymeric electrolyte (it has the same charge as that of the biological molecule), the biological molecule and the second polymeric electrolyte are present in a mixed state forming ion complex with the first polymeric electrolyte and, hence, immobilized on the porous carbon membrane.

As long as the biological molecules to be immobilized has a property where they have positive charge or negative charge in the solution (a) or the solution (b), they may be any of protein such as enzyme, antigen and antibody; nucleic acid such as oligonucleotide, polynucleotide and gene; lipid; and carbohydrate. Specifically, the examples include those exemplified in the previously-described section of <Immobilization of the biological molecule> as a biological molecule immobilizable utilizing the electrostatic interaction.

Although a polymeric electrolyte other than the biological molecule to be immobilized (i.e., the biological molecule to be immobilized for the purpose of exerting its function) may be a polymeric compound possessing a functional group capable of bearing positive charge or a polymeric compound possessing a functional group capable of bearing negative charge, it is preferably polycation or polyanion possessing a plurality of its functional groups.

The polycations include polymeric compounds possessing a plurality of functional groups capable of bearing positive charge, for example amino group. Specific examples include polyethylenimine, polyallylamine, polyvinylpyrrolidone, polylysine, polyvinylimidazole, and polyvinylpyridine.

The polyanions include polymeric compounds possessing a plurality of functional groups capable of bearing negative charge, for example carboxylic acid group and sulfonic acid group. Specific examples include a synthetic polymer such as polyacrylic acid, polymethacrylic acid, polystyrene sulfuric acid, and polymaleic acid; polysaccharide such as carboxymethylcellulose sodium and fucoidan; and nucleic acid such as DNA and RNA.

These polymeric electrolytes are preferably soluble in water or an organic solvent, particularly in water. They may also be a copolymer without restricted to a homopolymer.

Further, polymeric electrolytes which may be used herein are those in which a metal complex such as ferrocene, osmium bipyridines, or ruthenium bipyridines is introduced into a polymer by covalent bond or coordinate bond.

Although the solution containing the biological molecule or other polymeric electrolyte is generally aqueous solution, it may also contain a water-compatible organic solvent (methanol etc). The pH of the aqueous solution is preferably adjusted to hold its electrical charge state. It is possible to adjust the pH by using a dissociable functional group such as amino group and carboxylic acid group in the polymeric electrolyte, or it is also possible to adjust the pH by a buffer solution component such as phosphate.

Although the concentration of the solution to be used for the immersion is not particularly restricted, a level of 100 mg to 0.1 mg/ml, usually 1 mg/ml is used for the biological molecule solution. Although the concentration of other polymeric electrolyte is likewise a level of 100 mg to 0.1 mg/ml, it is also possible to use a polymer as itself when the polymeric electrolyte is a liquid polymer.

When a plurality of polymeric electrolytes is present in a single solution, they are preferably selected from those having the same electrical charge in the solution. A mono-molecular electrolyte compound may also be contained together with the polymeric electrolyte. For example, mono-molecular anions such as ferricyanide ion and polyanions such as polyacrylic acid may be contained to form polyion complex in which the mono-molecular anions are incorporated, whereby simultaneous immobilization is attained. The mono-molecular electrolyte compound as above, also is preferably selected from those having the same electrical charge in the solution.

In the immersion step of the porous carbon membrane (the sub-steps (a) and (b)), the enough amount of the solution to immerse the porous carbon membrane is firstly provided, and then the porous carbon membrane may be immersed. Although the immersion time is not particularly restricted, it is preferably 1 to 60 minutes for example. During the immersion treatment, it may be either still standing or shaken; shaking is more preferable for promoting diffusion into the pores.

To bring the solution into contact inside pores of the porous carbon membrane, an operation replacing inside of the pores is preferably added during the immersion, such as by deaerating inside of the pores once under a reduced pressure, followed by recovering to an ordinary pressure. Likewise, it is also desirable to promote the replacement in the pores in the solution during the immersion by adding a centrifuge operation to put the gravity to the entirety of the solution.

Although the temperature during the immersion is neither restricted, it is preferably 0° C. to 60° C., more preferably 0° C. to 30° C. for using an aqueous solution and biological molecule.

After, by this way, the porous carbon membrane is immersed into the solution (a) or the solution (b), it is immersed into the solution containing the polymeric electrolyte with the opposite electrical charge. That is, by immersing into the solution (b) after immersing into the solution (a), and immersing into the solution (a) after immersing into the solution (b), the biological molecule (together with the second polymeric electrolyte if present) and the first polymeric electrolyte with the opposite electrical charge are alternately stacked while forming the ion complex.

After these immersion steps, the porous carbon membrane is preferably washed before immersing into the solution of the polymer with the opposite electrical charge. For such washing, the entire membrane may be only washed with purified water or buffer solution; it is also preferable to add an operation to remove the solution from the membrane by absorbing and removing the solution in the membrane on a water-absorbing sheet such as a filter paper or suction filtration of the membrane after washing. It is also possible to reduce the contamination of the polymer solution with the opposite electrical charge by the immersion treatment with purified water before immersing into the polymer solution with electrical charge. By adding these washing steps, it is possible to prevent the occurrence of aggregation between the positive and negative polymeric electrolytes and to uniformly immobilize the biological molecule on the surface inside the pores.

Although the number of alternately stacking is not particularly restricted, it is once to twenty times, preferably once to ten times.

It is also preferable to adjust the pHs of the solution (a) and the solution (b) so that the polymeric electrolyte previously stacked on the membrane keeps the state of its electrical charge, in addition to that the polymeric electrolyte in the solution keeps a prescribed state of the electrical charge. For this purpose, the solution (a) and the solution (b) are preferably adjusted to have almost equal pH values.

It is also preferable that the anion group-introduced porous carbon is treated with an organic solvent solution of polycation which is soluble in the organic solvent such as polyethylenimine to form the first polyion complex. This is because coating the surface in the pores is promoted by using an organic solvent with low viscosity.

In a preferable embodiment, the biological molecule-immobilized carbon membrane produced by the layer-by-layer stacking method like this also holds the three-dimensional cancellous structure even after the immobilization of the biological molecule, and it has the air permeability of 1 to 2,000 sec/100 cc, particularly preferably 10 to 2,000 sec/100 cc. This is based on the fact that the present invention can perform the immobilization onto the pore surfaces with less occurrence of aggregation of the biological molecule by selecting appropriate conditions.

By the layer-by-layer stacking method like this, a larger amount of the biological molecule can be immobilized on the surface in the pores of the porous carbon membrane than the conventional method while keeping the air permeability. It is further possible to immobilize on the porous carbon membrane a compound that works together with the biological molecule such as a mediator compound as described in the section of bio-sensors. Therefore, further improvement of the membrane's functionality are achieved and wider applications are possible.

<Application of the Biological Molecule-Immobilized Porous Carbon Membrane>

In the present invention, it is possible to immobilize various biological molecules on the porous carbon membrane with a large specific surface having fine pores mutually connected each other The invention provides the effects of increase in sensitivity when used for a sensor and increase in output when used for an electrical generation element are obtained. It is also useable for an application with a purpose of uniform dispersion.

By using the layer-by-layer stacking method, it is particularly possible to immobilize a larger amount of the biological molecule in usable state on the porous carbon membrane. Comparing with a membrane immobilized by the single layer stacking method, a higher-sensitive sensor is realized in the application of a sensor, and a higher output is realized in the application of a bio-fuel cell. Since, furthermore, it is easy to immobilize other compounds such as a mediator in addition to the biological molecule by the layer-by-layer stacking method, the addition of mediator in the measurement sample is not necessary in the application of a sensor, and a simple layer structure is possible in the application of a bio-fuel cell.

Application in the Field of Sensor:

The functional carbon membrane on which a suitable enzyme, antigen, or antibody is immobilized in the present invention may be used as an electrode for a sensor. In the sensor of the present invention, when the enzyme-immobilized porous carbon membrane obtained in the present invention is contacted with a measurement object, a mediator molecule is reduced while the substrate is oxidized. The current value for the anodic oxidation of this reduced mediator is measured by the amperometry method, and the concentration of the measurement object is quantitatively determined. The measurement object compound includes those capable of becoming an enzyme's substrate: glucose can be measured when glucose oxidase or glucose dehydrogenase is immobilized, and ethanol can be measured when alcohol dehydrogenase is immobilized. For this purpose, the biological molecule to be immobilized is preferably glucose oxidase, glucose dehydrogenase, fructose dehydrogenase, or alcohol dehydrogenase.

Although voltage to be applied at the measurement by the amperometry method depends on the mediator to be used, for example, 0.1 V to 0.8 V is used. The measurement is possible if the enzyme-immobilized porous carbon membrane contacts the measurement object substance, and therefore it is also possible to use the flow-injection analysis (hereafter, abbreviated as FIA) where the measurement is conducted while the measurement object is allowed to flow through the porous carbon membrane.

For example, the functional membrane on which glucose oxidase or PQQ-dependent glucose dehydrogenase is immobilized by the above-described method may be used as the electrode for the glucose sensor. The most preferable immobilization method is the method for immobilizing by the electrostatic interaction, particularly the method for immobilizing by the layer-by-layer stacking method. It is also possible to immobilize by the physical interaction with the cross-linking using glutaraldehyde.

Since fluid can flow through inside of fine pores with large total surface area in the functional membrane in the present invention, the substantial amount of the enzyme capable of involved in the reaction can be increased, and as a result, the high-sensitive sensor can be obtained.

To construct the sensor, a known structure may be adopted for a part except the enzyme-immobilized electrode. For example, a known mediator such as hydroquinone, potassium ferricyanide, ferrocenecarboxylic acid, N-(2-chloro-1,4-naphthoquinone) phthalimide, 2,2,4-trimethyl-2,3-dihydro-1H-1,5-benzodiazepine, 2-methyl-1,4-naphthoquinone, 2-amino-3-carboxy-1,4-naphthoquinone, osmium (III)-(bipyridyl)-2-imidazolyl-chloride is added as needed.

In addition, it is also possible to immobilize the mediator when the layer-by-layer stacking method is used for immobilizing the biological molecule.

As the first example, an example is shown to immobilize glucose oxidase together with the mediator on the porous carbon membrane by the layer-by-layer stacking method. Table 1 shows an example for the solution (a) and the solution (b) used as layer-by-layer stacking.

TABLE 1

| | Polymeric electrolyte etc. | Polymeric electrolyte etc. |
|---|---|---|
| | in the solution (a) | in the solution (b) |
| | (cation-type) (The solution | (anion-type) (The solution |
| Material | is adjusted to pH 5.) | is adjusted to pH 5.) |
| Enzyme | | Gox |
| Mediator | PVI-dmeOs | |

Gox: glucose oxidase
PVI-dmeOs: poly(1-vinylimidazole) complexed with Os-(4,4-dimethylbipyridine)₂Cl

Since glucose oxidase bears negative charge at near-neutral pH range, the porous carbon into which the anion group is introduced is first immersion-treated with the polycation solution, followed by the treatment with the glucose oxidase solution. By serially repeating this operation, the immobilization by layer-by-layer stacking progresses. For the polycation as shown in the table, the mediator may be immobilized together with the enzyme by using, for example, the polycation with which the metal complex is coordinated (such as poly(1-vinylimidazole) complexed with Os-(4,4-dimethylbipyridine)₂Cl etc.).

As the second example, an example is shown to immobilize PQQ-dependent glucose dehydrogenase together with the mediator on the porous carbon membrane by the layer-by-layer stacking method. Table 2 shows an example for the solution (a) and the solution (b) used as layer-by-layer stacking.

TABLE 2

| | Polymeric electrolyte etc. | Polymeric electrolyte etc. |
|---|---|---|
| | in the solution (a) | in the solution (b) |
| | (cation-type) (The solution | (anion-type) (The solution |
| | is adjusted to pH 6.0.) | is adjusted to pH 6.0.) |
| Enzyme | PQQ-GDH | |
| Mediator | PVI-dmeOs | |
| Other | | Polyacrylic acid |
| polymeric | | |
| electrolyte | | |

PQQ-GDH: PQQ-dependent glucose dehydrogenase
PVI-dmeOs: poly(1-vinylimidazole) complexed with Os-(4,4-dimethylbipyridine)₂Cl

Since PQQ-dependent glucose dehydrogenase bears positive charge at near-neutral pH range, it may be immobilized by the layer-by-layer stacking in combination with the polyanion such as polyacrylic acid. In this example, the solution (a) contains the mixture of PQQ-dependent glucose dehydrogenase and the polycation with which the metal complex is coordinated (such as poly(1-vinylimidazole) complexed with Os-(4,4-dimethylbipyridine)₂Cl etc.) to co-immobilize the mediator together with the enzyme.

Although the PVI-dmeOs is used for the mediator immobilized onto the carbon membrane in the above-described examples, a monomolecular electrolyte compound, not only the polymeric electrolyte-type complex, may also be available as long as it mediates the electron transfer between the enzyme's active center and the electrode. As the polymeric electrolyte-type, ferrocenes, ruthenium complexes, and so on may be used. Not limited within complexes, those containing covalently-bound quinone-base compound may also be used.

Application in the Field of Bio-Fuel Cell:

A bio-fuel cell involves an oxidation reaction of fuel such as glucose, fructose, or ethanol as the fuel on an anode, and a reduction reaction of oxygen on a cathode.

The anode-side electrode preferably comprises enzyme capable of oxygenating a substrate such as glucose and the like, and optionally coenzyme and mediator, each of which are immobilized on the anode. The oxidation reaction of the substrate progresses on the anode to extract electrons outside the system.

For the anode-side, therefore, those having the identical structure to that of the previously-described sensors may be basically used. Since the magnitude of response electrical current influences the ability as the cell, those in which the enzyme is immobilized by the layer-by-layer stacking method have a particular advantage.

For the cathode-side, therefore, those in which bilirubin oxidase, laccase and so on as well as a mediator as needed are immobilized may be used (to be described later). Alternatively, an electrode supporting a metal catalyst such as platinum may also be used. When the enzyme-immobilized electrode is used for the cathode, the cell may be constructed by bringing the anode and cathode into contact with the identical fuel solution. When the electrode supporting the metal catalyst such as platinum is used for the cathode, the cell may be constructed by bringing the anode and cathode into contact through a proton conductor, while contacting the cathode with the fuel solution and contacting the anode with air or oxygen. The proton conductor includes a cation exchange resin such as Nafion (a trade name by DuPont).

An example is explained to construct the bio-fuel cell cathode by the biological molecule-immobilized carbon membrane of the present invention. For this purpose, the biological molecule to be immobilized is preferably bilirubin oxidase or laccase. It is also possible to immobilize the mediator.

As an example, an example is shown to immobilize bilirubin oxidase together with the mediator on the porous carbon membrane by the layer-by-layer stacking method. Table 3 shows an example for the solution (a) and the solution (b) used as layer-by-layer stacking.

TABLE 3

| | Polymeric electrolyte etc. | Polymeric electrolyte etc. |
|---|---|---|
| | in the solution (a) | in the solution (b) |
| | (cation-type) (The solution | (anion-type) (The solution |
| | is adjusted to pH 7.0.) | is adjusted to pH 7.0.) |
| Enxyme | | BOD |
| Mediator | | K₃[Fe(CN)₆] |
| Other | PAA | |
| polymeric | | |
| electrolyte | | |

BOD: bilirubin oxidase
PAA: polyallylamine

Since bilirubin oxidase bears negative charge at near-neutral, the porous carbon into which the anion group is introduced is first immersion-treated with the polycation solution, followed by the treatment with the bilirubin oxidase solution. By serially repeating this operation, the immobilization by layer-by-layer stacking progresses. For the polycation as shown in the table, for example, polyallylamine etc. may be used. By mixing ferricyanide ion serving as the mediator together with bilirubin oxidase into the solution (b), it may be immobilized at the same time together with bilirubin oxidase. Alternatively, it is possible to immobilize the ferricyanide ion by immersing into the ferricyanide ion solution after the immobilization treatment.

Instead of the ferricyanide ion, it is also possible to use a metal cyano complex such as tungsten and molybdenum. Alternatively, poly(1-vinylimidazole) complexed with Os-(4,4-dimethylbipyridine)₂Cl and the like may be also used as the polycation in the solution (a).

As above, the bio-fuel cell does not need noble metal catalysts and furthermore it can work even without a separator if a mediator-less construction or a construction immobilizing the mediator on the electrode is adopted. Therefore, an extremely simple construction can be realized. Since fluid can flow through a large surface area inside of the fine pores in the functional carbon membrane having immobilized enzyme according to the present invention, the substantial amount of the enzyme capable of involved in the reaction can be increased, and as a result, the high-output fuel cell can be obtained.

Although the concentration of the fuel solution is not particularly restricted, for example, it is 0.01 mol/L to 1 mol/L. The fuel solution may be either still standing or a circulation type.

Application as the Carrier:

The functional carbon membrane of the present invention may be utilized as a carrier to provide a place of reaction even other than the above-described application of the electrode. For example, the functional carbon membrane having immobilized biological molecules may be utilized in catalyst application.

For example, ferritin is a protein containing an iron oxide nanoparticle, and it is possible to replace the iron oxide nanoparticle with cobalt or palladium. The functional carbon membrane obtained by immobilizing the protein containing such metal element onto the porous carbon membrane carries the metal element uniformly and with high density. When organic materials are removed by calcination if needed, the functional carbon membrane carrying only an inorganic component such as metals and metal oxides on the carbon membrane surface may be obtained. For example, the calcined porous carbon membrane including the immobilized ferritin in which iron oxide is replaced with palladium may be utilized as a catalyst. The calcined porous carbon membrane including the immobilized ferritin in which iron oxide is replaced with cobalt is expected to be utilized as a recording material.

### EXAMPLES

### Referential Example 1

### Production of the Porous Polyimide Film

Using biphenyltetracarboxylic dianhydrides (s-BPDA) as a tetracarboxylic acid component and p-phenylenediamine (PPD) as a diamine component, the monomer components were dissolved in NMP so that their total weight was 8 wt %, and polymerization was carried out to give the solution of polyimide precursor having a logarithmic viscosity (30° C., concentration; 0.5 g/100 mL NMP) of 3.3.

The obtained polyimide precursor solution was flowed and cast in its thickness of about 400 μm, and further to its upper part NMP was uniformly applied by using a doctor's knife and they were left at rest for one minute, followed by immersing that stacking for 8 minutes into a congelation bath in which methanol and isopropanol were well mixed in a volume ratio of 1 vs. 1 to replace the solvent, whereby causing the precipitation of the polyimide precursor and forming the porous structure. After the porous film of the precipitated polyimide precursor was immersed in water for 15 minutes, it was exfoliated from a substrate and the heat treatment was carried out in the air at a temperature of 430° C. for 20 minutes in the state of fixed onto a pin tenter. The imidization ratio of the polyimide porous film was 80% and it has pores communicating in the direction of film cross-section.

### Referential Example 2

### Production of the Porous Carbon Membrane

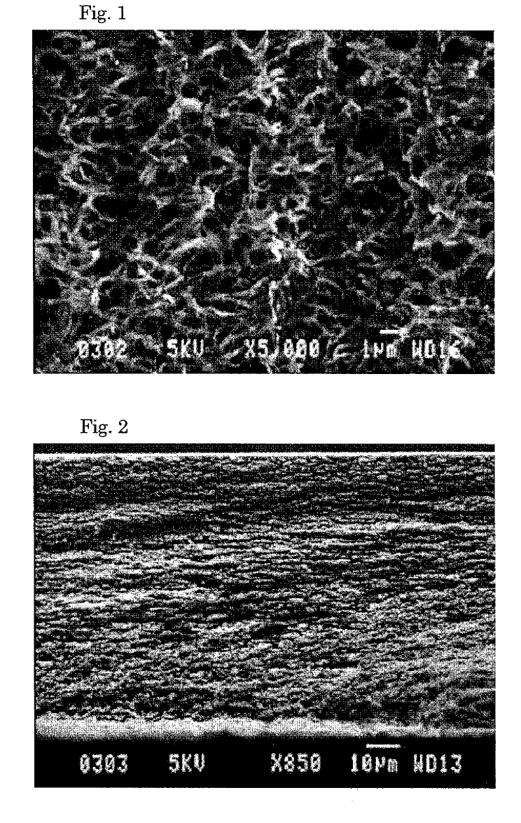

The porous polyimide film produced by Referential Example 1 was carbonized under nitrogen gas stream at a temperature of 1600° C. to give the porous carbon film having a film thickness of about 80 μm, an air permeability of 126 sec/100 ml, a vacancy ratio of 40%, and a mean pore diameter of 0.13 μm. The BET specific surface area by nitrogen adsorption measurement was also 13.8 m²/g. FIG. 1 shows the surface SEM image of the obtained carbon membrane, and FIG. 2 shows its cross-section SEM image.

The membrane properties were measured in accordance with the following method.

(1) Air Permeability

It was measured in accordance with JIS P8117. As a measurement instrument, B-type Gurley densometer (made by Toyo Seiki) was used. The sample membrane is tightened over a round hole with a diameter of 28.6 mm and area of 645 mm², the air inside of the cylinder is allowed to pass through from the test-round-hole zone to outside by the weight of 567 g of inner cylinder. The time allowing 100 cc air to pass through was measured to be the air permeability (Gurley value).

(2) Vacancy Ratio

It was calculated by determining the true density and the weight method.

(3) Mean Pore Diameter

The porous membrane was assessed based on the bubble point method (ASTM F316, JISK 3832). By using the perm-porometer of the Porous Materials Inc., the penetration-path distribution of the porous membrane was measured by the bubble point method, and the mean pore diameter was obtained by back calculation from the mean flow rate.

(4) Specific Surface Area

It was calculated by the BET method.

(5) Pore Diameter Distribution

It was calculated by the Dollimore-Heal (DH) method by utilizing the nitrogen adsorption isothermal curve.

### Example 1

### Oxidation Treatment of the Porous Carbon Membrane

The porous carbon membrane 2.00 g was measured off into a 300 ml flat-bottom separable flask, and normal concentration nitric acid 100 ml was added and they were gently refluxed for 8 hours. Then, they were collected by filtration, and washing with purified water was repeated until the pH of the washing solution became neutral. Then, drying was carried out under reduced pressure. Since a method for evaluating the oxidation degree of carbon has not been generally established, the elemental analysis used for usual analyses for organic compounds was adopted. An increase in oxygen contents was observed responding to the nitric acid treatment when such analysis was applied for this sample. Therefore, this was used as one of the evaluation means of oxidation degree. Table 4 shows those results.

TABLE 4
Results of the elemental analysis.

| | Nitric acid concentration | | | | |
|---|---|---|---|---|---|
| Element | untreated | 69% | 35% | 20% | 10% |
| H | N.D. | 1.33 | 0.96 | 0.34 | N.D. |
| C | 99.98 | 70.43 | 82.37 | 94.93 | 98.95 |
| N | N.D. | 0.63 | 0.51 | 0.48 | N.D. |
| O | N.D. | 25.47 | 15.36 | 3.06 | 0.80 |

ND: below detection limit

Table 5 also shows the results of the surface elemental analysis by XPS.

TABLE 5
Surface elemental concentration for the samples (atomic %).

| Sample name | C | N | O | S | Cl |
|---|---|---|---|---|---|
| i) Blank | 98.2 | 0.48 | 1.33 | | |
| ii) Treated with | 92.2 | 0.97 | 6.81 | | |
| nitric acid | | | | | |
| iii) After | 84.5 | 8.99 | 5.35 | 0.35 | 0.86 |
| polyethylenimine | | | | | |
| immobilization | | | | | |

In the table, those treated with nitric acid were treated with 35% nitric acid concentration.

### Example 2

### Introduction of Polyethylenimine onto the Porous Carbon Membrane Surface

About 1.0 g of the porous carbon membrane oxidation-treated with 35% nitric acid was measured off into a 300 ml flat-bottom separable flask, and DMF 0.2 ml and thionyl chloride 20 ml were added and they were gently refluxed for 4 hours in a draft on a hot plate after a cooling pipe was equipped. After cooled to room temperature, thionyl chloride was removed by decantation and the membrane was dried under reduced pressure.

Then, polyethylenimine (hereafter, abbreviated as PEI (Mn 600, Mw 800)) 20 ml was added and placed in a desiccator. The pressure in the desiccator was reduced to 0.1 Mpa or lower while it was heated up to about 60° C. to reduce the viscosity of polyethylenimine and increase its fluidity. The reduced pressure state was kept for about 10 minutes until occurrence of fine bubbles from the porous membrane disappeared. Then, it was recovered to ordinary pressure and the same operation was repeated three times to replace the air inside pores with PEI. Then, it was kept for 4 days at 40° C.

After completion of the reaction, the membrane was repeatedly washed with warm water to remove unreacted PET. The elemental analysis was conducted for the porous carbon membrane before and after the PEI treatment. Table 6 shows that result. A noticeable increase of N element amount after the PEI treatment was observed. The results of the surface elemental analysis by XPS is also shown in Table 5.

TABLE 6
Elemental analysis (wt %).

| | H | C | N | O |
|---|---|---|---|---|
| Before the PEI | 1.56 | 92.23 | 0.41 | 4.99 |
| treatment | | | | |
| After the PEI | 0.65 | 93.85 | 1.28 | 3.43 |
| treatment | | | | |

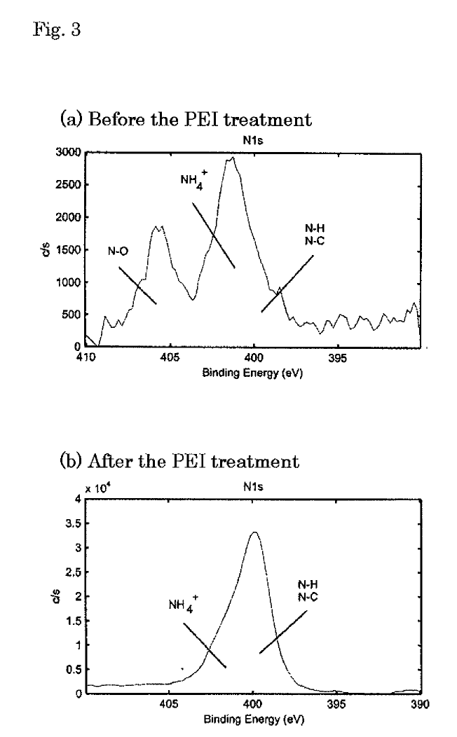

As furthermore shown in FIGS. 3 (a) and (b), the introduction of the PEI onto the carbon membrane surface was affirmed due to the observation of existence of NH bond by the PEI treatment in comparison with the XPS spectrum of the porous carbon membrane before and after the PEI treatment.

### Example 3

### Immobilization of the Metal Nanoparticle-Carried Protein (Ferritin)

7 ml of commercially available ferritin (Sigma F-4503; ferritin concentration 76 mg/me was dialyzed for 16 hours in a dialysis tube (Wako, Cellulose tubing, Small Size 24) with distilled water as an external solution at 4° C. After the dialysis, it was collected from the tube and diluted to 10 ml with distilled water (ferritin concentration 53 mg/ml). 0.1 M succinic acid buffer solution (pH 4.5) 1 ml was added to 9 ml of the desalination-treated ferritin aqueous solution (53 mg/ml). Since the pH changed to about 5 by measuring the pH after the addition, the pH was re-adjusted to 4.5 with dilute hydrochloric acid.

The porous carbon membrane 20 mg oxidation-treated with 35% nitric acid in Example 1 was transferred into a 30 ml sample tube, and the above-described ferritin solution 5 ml was added. A vessel was placed in a desiccator and the pressure was reduced to 0.1 MPa or lower. The reduced pressure state was kept for about 10 minutes until occurrence of fine bubbles from the porous membrane disappeared. Then, it was recovered to ordinary pressure and the re-reduction of pressure was repeated three times, followed by gently shaking at 4° C. on a shaker for 24 hours. Then, the carbon membrane was collected and repeatedly washed with purified water, followed by drying in a vacuum desiccator.

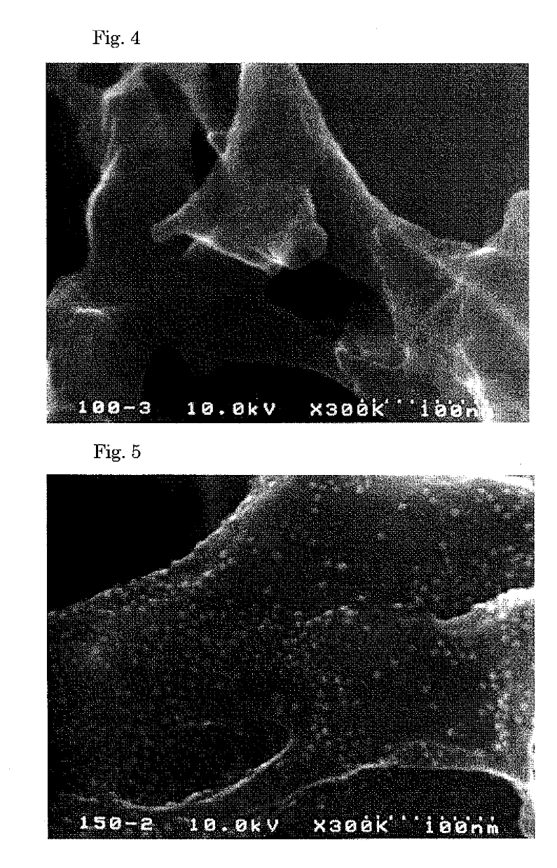

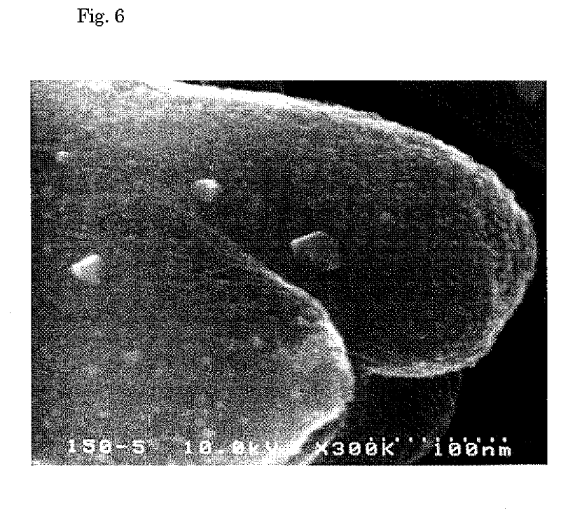

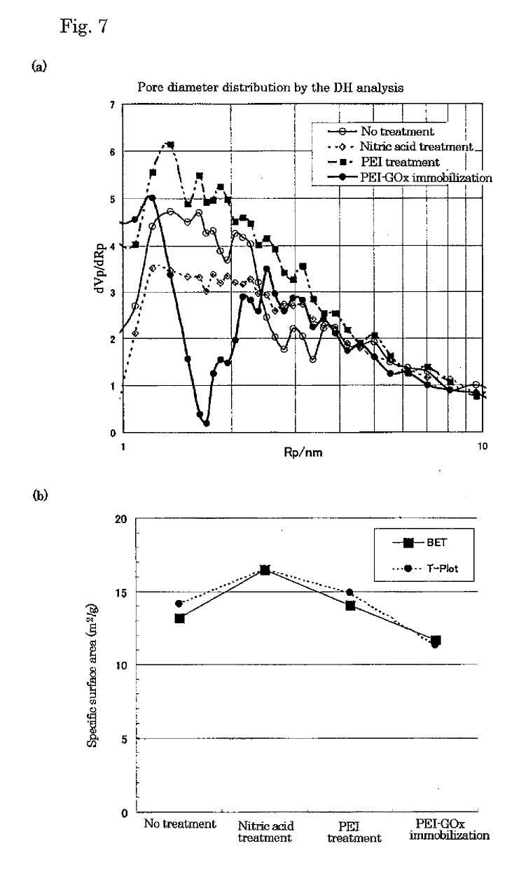

After drying the membrane, the amount of Fe₃O₃ was quantitatively determined by the X-ray fluorescence analysis (XRF analysis). The SEM observation was also carried out. Then, the membrane was calcinated by raising temperature from room temperature at the rate of 10° C./min and keeping at 500° C. for 1 hour under N2 stream (100 ml/min), and was allowed to be cooled. For the calcined samples, the SEM observation and X-ray fluorescence analysis were also carried out. Table 7 shows the quantitative results by the X-ray fluorescence analysis before the immobilization, after the immobilization and after the calcination, respectively, FIG. 4, FIG. 5 and FIG. 6 also show the respective SEM photograph images.

TABLE 7
Quantitative determination of the elemental composition by
the X-ray fluorescence analysis (XRF analysis).

| | Before | After | After |
|---|---|---|---|
| | immobilizing | immobilizing | calcining |
| Fe(wt %) | 0.0284 | 2.09 | 5.37 |
| Fe₂O₃(wt %) | 0.0406 | 2.99 | 7.68 |

(Quantitative analysis FP method: measuring the mean value of element amount of diameter 25 mm.)

(Fe Analysis in the Cross-Sectional Direction of the Membrane)

For the Immobilized Sample, the SEM-EDS Measurement and the EPMA analysis were carried out to quantitatively determine the amount of the immobilized enzyme in the cross-sectional direction. Since the porous carbon membrane before immobilizing the enzyme scarcely contains iron element and the immobilized enzyme ferritin is a protein containing iron oxide nanoparticle, the iron element amount is proportional to the amount of existing ferritin.

Production of the Samples:

After embedding the ferritin-immobilized porous carbon membrane into epoxy resin, a cross-section was formed by the microtome processing, and Pt-coating was performed to reduce electrostatic charge during SEM observation, and thus the sample was produced.

SEM-EDS Measurement:

By using the produced sample, the cross-sectional direction image was taken by the FE-SEM (S-4200 made by Hitachi, field-emission-type scanning electron microscope) under an accelerating voltage of 3 kV (secondary electron image) and 15 kV (reflection electron image). The presence of Fe element was confirmed and quantitatively determined by the EDS (used instrument: SIGMA Type II made by KEVEX [⁵B to ⁹²U]: accelerating voltage: 20 kV) from the EDS analysis of two respective sites with spot size of width 2 μm and height 1.5 μm at the membrane top surface (Top; about 5 μm from the surface), the intermediary part (medium; depth of about 20 μm from the both surfaces), and the membrane top surface (under; about 5 μm from the surface). Table 8 shows that result.

TABLE 8

| | Peak intensity (Cnts/s)*⁾ |
|---|---|
| Membrane top surface Top | 33.61, 36.05 |
| (about 5 μm from the surface) | |
| Intermediary part Medium | 16.09, 17.29 |
| (Depth of about 20 μm from the both | |
| surfaces) | |
| Membrane top surface Under | 22.51, 25.26 |
| (about 5 μm from the surface) | |

*⁾The distance between two sites of the measurement points was about 20 μm.

Since the EDX spectrum intensity is proportional to the element amount, it was demonstrated that the Fe element exists not only on the membrane surface (near outside) but also on the membrane inward (surface of the membrane inward).

EPMA (Electron Probe Micro Analyzer) Analysis:

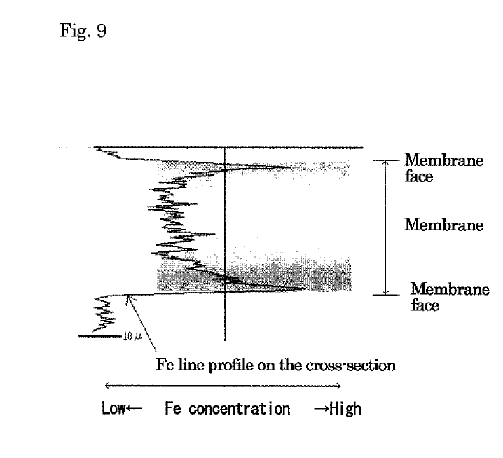

By using the same sample, the iron element distribution was measured by the EPMA analysis. By using the electron-ray analyzer JXA-8800R (wavelength-dispersive type) made by JEOL, the measurement was conducted by the condition of accelerating voltage of 15 kV, irradiation electrical current of 1.0×10⁻⁷ A, probe diameter of 5 μm. FIG. 9 shows the measurement result of Fe concentration on the cross-section by EPMA.

In the analysis result by the EPMA, it was also shown that Fe element is not unevenly present in the membrane surface (i.e., near outside), but it exists also in the inside of membrane. By the line profile, there is a layer with a high concentration of Fe at a position of about 5 μm from the surface, and in the intermediary part inside the position the Fe concentration is almost constant.

Hence, the biological molecules are significantly not unevenly distributed near outside the membrane, but they exist also in the inside of membrane with a sufficient portion in comparison with the outside.

### Example 4

### Immobilization of Glucose Oxidase: Immobilization by the Electrostatic Interaction

Glucose oxidase (made by Amano Enzyme, hereafter abbreviated as Gox) 50 mg was dissolved in 5 ml of 5 mM phosphate buffer solution (pH 7.0) to form an enzyme solution. The PEI-treated carbon membrane of 95 mg produced in Example 2 was placed in a 4 cm glass petri dish, and the enzyme solution was added so that the entire membrane soaked. Then, to replace the air in the inside of pores with the enzyme solution, the vessel was placed in a desiccator and the pressure was reduced by a vacuum pump, and after establishing well reduced pressure, it was recovered to ordinary pressure and the re-reduction of pressure was repeated three times. It was left at 4° C. overnight, and the carbon membrane collected and repeatedly washed with purified water was dried under reduced pressure in a desiccator. The obtained membrane was provided for the subsequent measurement of the Gox activity. Until the measurement, it was stored at −20° C.

Gox Activity Measurement:

The following reagents were prepared.

A. Aminoantipyrine solution (4 mg/ml): It was prepared by that 0.2 g of aminoantipyrine was dissolved in purified water 20 ml, followed by adjusting its volume to 50 ml.
B. Phenol solution (50 mg/ml): It was prepared by that 2.5 g phenol was dissolved in purified water 20 ml, followed by adjusting its volume to 50 ml.
C. Peroxidase solution: It was prepared by that 1,250 purpurogallin-units of peroxidase (SIGMA) was dissolved in 50 ml of distilled water (after preparation, it was stored on an ice bath).
D. 0.1 M phosphate buffer solution (KH₂PO₄—NaOH, pH 7.0):
E. Phenol buffer solution: It was prepared by that 0.13 g of KH₂PO₄ was dissolved in 80 ml of distilled water and 3 ml of the above-described B, phenol solution was added, followed by adjusting pH 7.0 with 1 N NaOH and its volume to 100 ml.
F. Substrate solution: It was prepared by that 5.0 g of D-glucose was dissolved in 50 ml of distilled water.

Several milligrams of the carbon membrane pieces was precisely measured off into a 30 ml sample tube, and 10.0 ml of the phenol buffer solution (E), 2.5 ml of the peroxidase solution (C), and 0.5 ml of the aminoantipyrine solution (A) were added, followed by shaking incubation for 5 minutes in a thermostatic chamber at 30° C. Then, the reaction was initiated by adding 2.5 ml of the substrate solution (F) warmed to 30° C. in advance.

While vigorously shaking, 1 ml of sample was collected each time after 2 minutes and 10 minutes, and an absorbance at 500 nm was quickly measured. After the measurement, the reaction solution was removed by decantation and washed with distilled water. Then, the measurements were repeated.

As its results, after the measurement and washing were repeated four times, 0.044 U of the enzyme activity per the immobilized porous carbon membrane 1 mg was observed in the fifth measurement.

FIGS. 7 (a) and (b) show the pore distribution and surface area for the untreated, treated with nitric acid, PEI-treated, PEI-treated and GOX-immobilized porous carbon membrane.

### Example 5

### Immobilization of PQQ-dependent Glucose Dehydrogenase: Immobilization by the Electrostatic Interaction

PQQ-dependent glucose dehydrogenase (made by Amano Enzyme, hereafter abbreviated as GDH) 50 mg was dissolved in 5 ml of 5 mM phosphate buffer solution (pH 7.0) to form an enzyme solution. 100 mg of the nitric acid-oxidized carbon membrane oxidation-treated with 35% nitric acid was placed in a 4 cm glass petri dish, and the enzyme solution was added so that the entire membrane soaked. Then, to replace the air in the inside of pores with the enzyme solution, the vessel was placed in a desiccator and the pressure was reduced by a vacuum pump, and after establishing well reduced pressure, it was recovered to ordinary pressure and the re-reduction of pressure was repeated three times. It was left at 4° C. overnight, and the carbon membrane collected and repeatedly washed with purified water was dried in a desiccator under reduced pressure, and was provided to the subsequent measurement of the GDH activity. Until the measurement, it was stored at −20° C.

GDH activity measurement:

The following reagents were firstly prepared.

### A. 3-(N-Morpholino) Propane Sulfonic Acid (Hereafter Abbreviated as MOPS) Buffer Solution:

2 mM CaCl₂ was added to 20 mM MOPS (pH 7).

### B. PMS Solution:

6.13 mg PMS (phenazine methosulfate)/1 ml deionized water was prepared (light blocked storage).

### C. DCIP Solution:

1.3 mg DCIP (dichloroindophenol)/1 ml deionized water was prepared (light blocked storage).

### D. Glucose Solution:

1.2 M glucose solution was prepared.

The carbon membrane after the immobilization treatment of enzyme was precisely measured off into a 20 ml screw tube, and 10.0 ml of the MOPS buffer solution, 0.2 ml of the PMS solution, and 0.2 ml of the DCIP solution were added, and the reaction was initiated by adding the substrate solution (1.0 ml glucose solution) and reciprocating-shaken at 160 rpm in a thermostatic chamber at 30° C. The reaction solution 1 ml was collected after one minute and 6 minutes after the above addition of the substrate solution, and an absorbance at 600 nm was measured in a UV cell.

After completing the measurement, the reaction solution was removed by decantation and the membrane was washed with distilled water and 0.05 M phosphate buffer solution (EDTA, pH 7.0). Then, the enzyme measurement operations were repeated to measure its activity.

As its results, after the measurement and washing were repeated nine times, 0.037 U of the enzyme activity per the immobilized porous carbon membrane 1 mg was observed in the tenth measurement.

(Measurement of the Air Permeability)

The air permeability of the enzyme-immobilized carbon membrane obtained in Example 5 was also measured in a similar manner to that of Referential Example 2, and as a result it was 220 sec/100 ml. This demonstrates that the mutual connection of the membrane pores sufficiently exists even after immobilizing the enzyme.

### Example 6

### Immobilization of Glucose Oxidase: Immobilization by the Physical Interaction (Cross-Linking Method)

### Example 6-1

Glucose oxidase (made by Amano Enzyme, hereafter abbreviated as Gox) 50 mg was dissolved in 1 ml of 10 mM phosphate buffer solution (pH 7.0) to form an enzyme solution, and 80 mg of BSA (bovine serum albumin) was separately dissolved in 1 ml of 10 mM phosphate buffer solution (pH 7.0) to prepare the BSA solution.

While stirring the mixture of the prepared enzyme solution 800 μl and the BSA solution 800 μl on a glass petri dish, 2.5% glutaraldehyde aqueous solution 400 μl was added to form the immobilizing enzyme solution. To the immobilizing enzyme solution, the porous carbon membrane was added so that the entire membrane soaked. Then, to replace the air in the inside of pores with the enzyme solution, the vessel was placed in a desiccator and the pressure was reduced by a vacuum pump, and after establishing well reduced pressure, it was recovered to ordinary pressure and the re-reduction of pressure was repeated three times. Then, the membrane was collected after it was left at room temperature for 3 hours, and it was dried under reduced pressure by a vacuum pump. Then, the carbon membrane was repeatedly washed with purified water, followed by drying in a desiccator under reduced pressure and providing for the GOX activity measurement.

After the measurements and washings were repeated four times like Example 4, 0.02 U of the enzyme activity per the immobilized porous carbon membrane 1 mg was observed in the fifth measurement.

As a comparative example, a similar treatment was conducted except that the glutaraldehyde aqueous solution and the BSA solution were not added, and instead 10 mM phosphate buffer solution (pH 7.0) 1.2 ml was added. The measurements and washings of the obtained carbon membrane were repeated four times, and the enzyme activity was not higher than 0.001 Upper the immobilized porous carbon membrane 1 mg in the fifth measurement, which means that the enzyme was not immobilized.

### Example 6-2

The cross-linking immobilization of the enzyme was attempted by using PEI instead of BSA used in Example 6-1. First, 50% aqueous solution of polyethylenimine (PEI; made by Aldrich (Mn 1800, Mw 2000)) was diluted five times with 10 mM phosphate buffer solution (pH 7.0) to form the PEI solution. 1,200 μl of 10 mM phosphate buffer solution (pH 7.0) was added to the mixture of the enzyme solution 800 μl and the PEI solution 100 μl, and then 2.5% glutaraldehyde aqueous solution 100 μl was added while stirring to form the immobilizing enzyme solution. Other treatments were conducted in a similar manner to those described in Example 6-1. After the measurements and washings of the obtained carbon membrane were repeated four times, 0.03 U of the enzyme activity per the immobilized porous carbon membrane 1 mg was observed in the fifth measurement.

(Measurement of the Air Permeability)

The air permeability of the enzyme-immobilized carbon membrane obtained in Example 6-2 was also measured in a similar manner to that of Referential Example 2, and as a result it was 205 sec/100 ml. This demonstrates that the mutual connection of the membrane pores sufficiently exists even after immobilizing the enzyme.

### Example 7

### Immobilization of PQQ-Dependent Glucose Dehydrogenase: Immobilization by the Physical Interaction (Cross-Linking Method)

PQQ-dependent glucose dehydrogenase (PQQ-GDH made by Amano Enzyme) 50 mg was dissolved in 1 ml of 10 mM phosphate buffer solution (pH 7.0) to form an enzyme solution, and polyethylenimine (PEI; made by Aldrich (Mn 1800, Mw 2000)) was separately diluted five times with 10 mM phosphate buffer solution (pH 7.0) to form the PEI solution.

On a glass petri dish, 1,200 μl of 10 mM phosphate buffer solution (pH 7.0) was added to the mixture of the enzyme solution 800 μl and the PEI solution 100 μl, and then 2.5% glutaraldehyde aqueous solution 100 μl was added while stirring to form the immobilizing enzyme solution. To the immobilizing enzyme solution, the porous carbon membrane was added so that the entire membrane soaked. Then, to replace the air in the inside of pores with the enzyme solution, the vessel was placed in a desiccator and the pressure was reduced by a vacuum pump, and after establishing well reduced pressure, it was recovered to ordinary pressure and the re-reduction of pressure was repeated three times. Then, the membrane was collected after it was left at room temperature for 3 hours, and it was dried under reduced pressure by a vacuum pump. Then, the carbon membrane repeatedly washed with purified water was dried in a desiccator under reduced pressure and provided for the PQQ-GDH activity measurement.

After the measurements and washings were repeated four times like Example 5, 0.025 U of the enzyme activity per the immobilized porous carbon membrane 1 mg was observed in the fifth measurement.

As a comparative example, a similar treatment was conducted except that the glutaraldehyde aqueous solution and the PEI solution were not added, and instead 10 mM phosphate buffer solution (pH 7.0) 1.2 ml was added. After the measurements and washings of the obtained carbon membrane were repeated four times, the enzyme activity was not higher than 0.001 U per the immobilized porous carbon membrane 1 mg in the fifth measurement, which means that the enzyme was not immobilized.

### Experimental Example 1 for the Sensor

Electrochemical measurement of the porous carbon membrane having immobilized enzymes thereon according to the present invention was carried out using three-electrode-system electrochemical cell. The three-electrode-system electrochemical cell was constructed of (i) a work electrode using the glassy carbon electrode with a diameter of 3 mm on which the porous carbon membrane physically adhered as a measurement object, (i) a reference electrode using Ag/AgCl electrode, and (iii) a counter electrode using Pt mesh electrode.

For the electrolyte solution when the immobilized enzyme was Gox, 10 ml of 0.2 M phosphate buffer solution (pH 7.0) containing 2 M KCl was used. Before the measurement, oxygen was purged with nitrogen gas for 20 minutes to replace it. 1 mM hydroquinone was also added as a mediator. When the immobilized enzyme was GDH, 10 ml of 0.02 M MOPS buffer solution (pH 7.0) containing 2 mM CaCl₂ was used. 0.1 mM ferrocenecarboxylic acid was added as a mediator.

The electrolyte solution containing the predetermined concentration of glucose was added the electrochemical cell, after stirring by a magnetic stirrer for 15 minutes, +0.3 V of voltage was applied and the electrical current value was measured after 2 minutes. During the measurement, the electrolyte cell was under nitrogen atmosphere.

Table 9 shows the result of the electrochemical measurement.

### Comparative Examples for the Sensor: Comparative Electrodes 1 and 2

For comparison with the porous carbon membrane of the present invention, electrodes were formed using a flat and smooth glassy carbon electrode (made by BAS, diameter 3 mm) on which the enzyme was cross-linked and immobilized with glutaraldehyde.

The experimental method was conducted in accordance with Humana Press “Immobilization of Enzymes and Cells” (1997), p 83 “Immobilization of Enzymes on Microelectrodes Using Chemical Crosslinking” as follows.

The enzyme 50 mg was dissolved in 1 ml of sodium chloride-containing phosphate buffer solution (5.3 mM phosphoric acid, 0.15 M sodium chloride, pH 7.2, hereafter referred as PBS buffer solution) to form an enzyme solution, and 80 mg BSA (bovine serum albumin) was separately dissolved in 1 ml of the PBS buffer solution to prepare the BSA solution.

While stirring the mixture of the prepared enzyme solution 50 μl and the BSA solution 250 μl, 2.5% glutaraldehyde aqueous solution 100 μl was added. Then, the sample 20 μl was immediately collected by a micropipette and applied on the electrode face of the glassy carbon electrode, and it was left at room temperature for 3 hours to form the thin film. Then, after immersed in the measurement solution for 30 minutes, the electrode was provided for the measurement.

The electrode using Gox as the enzyme was the comparative electrode 1, and the electrode using GDH as the enzyme was the comparative electrode 2. Table 9 shows the result of the electrochemical measurement.

TABLE 9

| Glucose | Electrical current value (μA) | | | |
|---|---|---|---|---|
| concen- | Gox-immobilized electrode | | GDH-immobilized electrode | |
| tration | Carbon membrane of | Comparative | Carbon membrane of | Comparative |
| (mM) | Example 4 was used. | electrode 1 | Example 5 was used. | electrode 2 |
| 0 | 6.4 | 4.38 | 4.84 | 0.035 |
| 0.01 | 6.56 | 4.49 | | 0.054 |
| 0.05 | 6.78 | | 4.73 | |
| 0.1 | 6.77 | 4.53 | 5.38 | 0.085 |
| 0.5 | 7.423 | | 6.91 | |
| 1 | 7.89 | 4.59 | 7.29 | 0.423 |
| 5 | 13.34 | 7.17 | 11.34 | 1.129 |
| 10 | 18.39 | 9.38 | 16.1 | 1.29 |
| 50 | 53.4 | 18.12 | 15.9 | 1.453 |

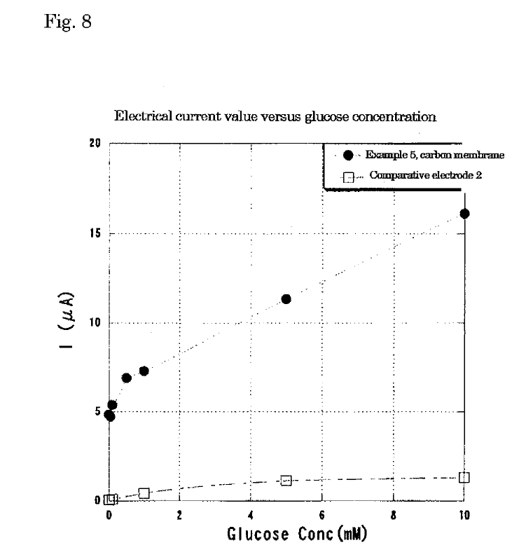

FIG. 8 also shows a graph in a range of a low concentration of glucose for the GDH immobilized electrode. From this result, the sensor of the present invention has a large electric current output and it is also suitable for sensing glucose with a low concentration.

In addition, these results indicate that the application for the bio-fuel cell is also obviously possible.

### Referential Example 3

### Synthesis of the Osmium Complex Polymer

### (i) Synthesis of the Os-Bipyridyl Type Complex

In accordance with the method of a reference (Inorg. Synth., 24, 291-299 (1986)), the synthesis was carried out by the following scheme.

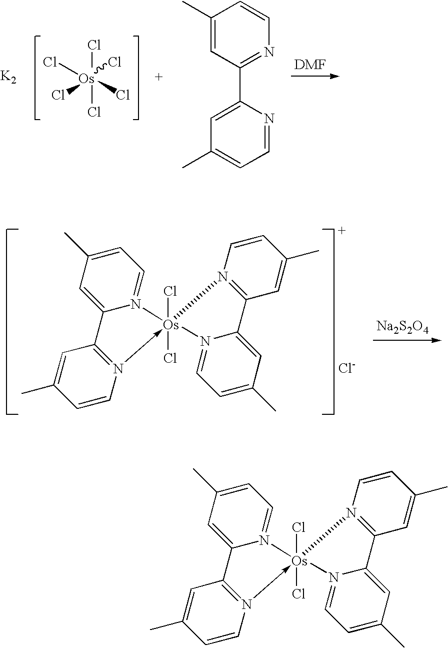

### (i) Synthesis of Os(4,4′-dimethylbpy)₂Cl₂

Potassium hexachloroosmate (IV) 0.225 g (0.46 mmol) and 2,2′-bi-4-picoline 0.18 g (1.0 mmol) (made by TCI) were added to a 20 ml round-bottom flask, dissolved in DMF 4 ml, and refluxed in a oil bath for one hour. After the reaction, they were allowed to cool for one hour to room temperature, followed by filtration. Ethanol 2 ml was added to the filtrate and they were added to diethylether 50 ml while vigorously stirring, and the resultant precipitate was filtrated off collected and dried to give 0.187 g of [Os(4,4′-dimethylbpy)₂Cl₂]Cl as black powder.

Analysis value: the calculated values for the dihydrate are C, 41.12; H, 4.03; N, 7.99 and the results of elemental analysis were C, 39.1; H, 4.19; N, 8.95.

Then, 0.18 g of [Os(4,4′-dimethylbpy)₂Cl₂]Cl was dissolved in DMF 3.6 ml and MeOH 1.8 ml in a 50 ml beaker. To the obtained black solution, sodium dithionate aqueous solution (Na₂S₂O₄ 0.36 g/water 36 ml) was intermittently added over about one hour. The reaction solution was observed to be slightly viscous and its color was changed from black to dark purple. Then it was continuously stirred for one hour in a ice bath, and the resultant precipitate was filtrated off and collected. The precipitate was washed with water 2 ml, MeOH 2 ml, and diethylether 2 ml, followed by drying under reduced pressure to give 104 mg of Os(4,4′-dimethylbpy)₂Cl₂ as the black targeted compound.

Analysis value: the calculated values for the dihydrate are C, 43.31; H, 4.24; N, 8.42 and the results of elemental analysis were C, 41.71; H, 3.68; N, 8.42.

### (ii) Synthesis of poly(1-vinylimidazole)

AIBN 0.5 g was added to a 100 ml two-necks Erlenmeyer flask, and the system inside was replaced with argon. Vinylimidazole (made by Aldrich) 6 ml was added through a septum. While stirring by a stirrer, it was heated in an oil bath. When the temperature of the bath reached 70° C., the polymerization progressed quickly and the liquid monomer turned into a yellow mucilaginous material. Then, the bath was kept at 70° C. for two hours, which was allowed to cool to room temperature. The solid was dissolved in MeOH 50 ml, and the mixture was added to 500 ml of acetone while vigorously stirring. The resultant pale yellow precipitate was filtrated off, collected and dried to give 2.25 g of poly(1-vinylimidazole) as the targeted compound.

### (iii) Synthesis of the Osmium Complex Polymer: poly(1-vinylimidazole) Complexed with Os-(4,4-dimethylbpy)₂Cl

Poly(1-vinylimidazole) 94 mg and ethanol 30 ml were added a 100 ml round-bottomed flask, and refluxed for 0.5 hours to be dissolved. Then, a solution in which Os(4,4′-dimethylbpy)₂Cl₂ 63 mg was dissolved in 10 ml ethanol was added at once, and the mixture was refluxed for 60 hours, After the completion of the reaction, the solvent was distilled away, and the residue was dissolved in about 15 ml methanol. The mixture was added to 150 ml of diethylether while vigorously stirring. The resultant precipitate was filtrated off, collected and dried to give 105 mg of the osmium complex polymer PVI-dmeOs as black powder of the targeted compound.

Analysis value: the calculated values for the PVI-dmeOs are C, 54.86; H, 5.95; N, 21.97 and the results of elemental analysis were C, 53.76; H, 5.89; N, 18.54.

### Example 8

### Immobilization of the Osmium Complex Polymer and Glucose Oxidase (Gox) onto the Porous Carbon Membrane by the Layer-By Layer Stacking Method

The polycation solution was prepared by dissolving the osmium complex polymer synthesized in Referential Example 3 in 10 mM acetate buffer solution (pH 5) at a concentration of 1 mg/ml.

The polyanion solution was prepared by dissolving glucose oxidase (220 u/mg made by Amano Enxyme) in 10 mM acetate buffer solution (pH 5) at a concentration of 1 mg/ml.

The porous carbon membrane obtained by the oxidation treatment of Example 1 was cut off in a about 2 cm square, and the following operations were carried out.

(1) The membrane is washed with pureed water while conducting suction filtration on a Kiriyama's funnel. After confirming that no water remained on the membrane, it was immersed into the polycation solution in wells of a polystyrene plate with six-wells, and repeatedly exposed to reduced pressure and ordinary pressure in a desiccator to replace the air in the membrane with the immobilization solution. Then, the entire plate is centrifuged at 1500 g for 10 minutes and left for 10 minutes while gently shaking on a plate shaker.

(2) Then, the membrane is collected and washed with purified water while conducting suction filtration on a Kiriyama's funnel. After confirming that no water remains on the membrane, it is immersed into purified water in a six-wells plate, and repeatedly exposed to reduced pressure and ordinary pressure in a desiccator to replace the air in the membrane with the immobilization solution. Then, the membrane is collected and washed with purified water while conducting suction filtration on a Kiriyama's funnel.

(3) After confirming that no water remains on the membrane, it is immersed into the polyanion solution in wells of a six-wells plate, and repeatedly exposed to reduced pressure and ordinary pressure in a desiccator to replace the air in the membrane with the immobilization solution. Then, the entire plate is centrifuged at 1500 g for 10 minutes and left for 10 minutes while gently shaking on a plate shaker.

Since, by the above-described operations 1 to 3, a single layer of the layer-by-layer stacking membrane is formed, the repeating number of this operation gives the number of layers. For example, the five-layer stacked membrane is obtained by repeating the operation five times.

Then, the membrane was collected and washed with purified water while conducting suction filtration on a Kiriyama's funnel. After confirming that no water remained on the membrane, it was dried in a vacuum desiccator and stored at −20° C.

### Example 9

### Immobilization of the Osmium Complex Polymer and PQQ-Dependent Glucose Dehydrogenase (GDH) onto the Porous Carbon Membrane by the Layer-By Layer Stacking Method

The same operations to those of Example 8 were carried out except for using the following compositions as the polycation solution and the polyanion solution.

The polycation solution for use was prepared by dissolving the osmium complex polymer and PQQ-dependent glucose dehydrogenase (4800 u/mg made by Amano Enzyme) in 10 mM phosphate buffer solution (pH 6) at each concentration of 1 mg/ml.

The polyanion solution for use was prepared by dissolving polyacrylic acid (average molecular weight 25,000) in purified water and adjusting to pH 6 with 1 mol/l NaOH, followed by diluting with purified water to the final concentration of 1 mg/ml.

(Measurement of the Air Permeability)

The air permeability of the enzyme-immobilized carbon membrane obtained in Example 9 was measured in a similar manner to that of Referential Example 2, and as a result it was 370 sec/100 ml. This demonstrates that the mutual connection of the membrane pores sufficiently exists after immobilizing the enzyme even by the layer-by layer stacking method.

### Example 10

### Immobilization of Potassium Ferricyanide and Bilirubin Oxidase onto the Porous Carbon Membrane by the Layer-By Layer Stacking Method

The same operations to those of Example 8 were carried out except for using the following compositions as the polycation solution and the polyanion solution.

The polycation solution used was a solution in which polyallylamine (PAA-15 made by Nittobo, average molecular weight 15,000) was dissolved in purified water and adjusted to pH 7 with 1 mol/l HCl, followed by diluting with purified water to the final concentration of 1 mg/ml.

The polyanion solution used was a solution in which bilirubin oxidase (hereafter abbreviated as BO, 2.43 u/mg made by Amano Enxyme) and K₃[Fe(CN)₆] were dissolved in 10 mM phosphate buffer solution (pH 7) at each concentration of 1 mg/ml.

(Measurement of the Air Permeability)

The air permeability of the enzyme-immobilized carbon membrane obtained in Example 10 was also measured in a similar manner to that of Referential Example 2, and as a result it was 213 sec/100 ml. This also demonstrates that the mutual connection of the membrane pores sufficiently exists even after immobilizing the enzyme by the layer-by layer stacking method.

### Example 11

### Immobilization of the Metal Nanoparticle-Carrying Protein (Ferritin) onto the Porous Carbon Membrane by the Layer-By Layer Stacking Method

The same operations to those of Example 8 were carried out except for using the following compositions as the polycation solution and the polyanion solution.

The polycation solution used was a solution in which polyallylamine (PAA-15 made by Nittobo, average molecular weight 15,000) was dissolved in purified water and adjusted to pH 7 with 1 mol/l HCl, followed by diluting with purified water to the final concentration of 1 mg/ml.

To prepare the polyanion solution, a commercially-available ferritin (76 mg/ml made by SIGMA) 5 ml was added into a semipermeable membrane and dialyzed to 1 L of an external solution (5 mM phosphate buffer solution pH 7) at 4° C. overnight while stirring. After the dialysis, the protein concentration in the solution was measured (the BCA method: 26 mg/ml) and diluted with the phosphate buffer solution (5mM pH 7) to give 1 mg/ml. Thus diluted solution was used for the polyanion solution.

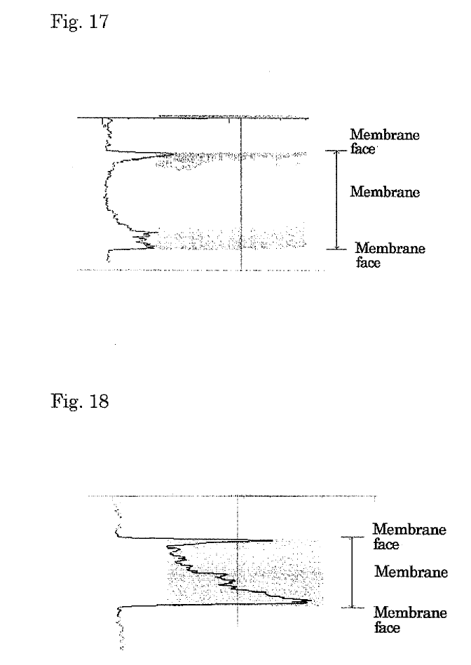

FIG. 17 shows the result of the EPMA analysis conducted in a similar manner to Example 3 for the five-layers-stacked membrane. Proportion of iron element unevenly existed near the membrane face was high.

### Example 12

### Immobilization of Potassium Ferricyanide and Bilirubin Oxidase onto the Large Porous Carbon Membrane by the Layer-By Layer Stacking Method

The same operations to those of Example 10 were carried out using the same compositions as the polycation solution and the polyanion solution of Example 10. The porous carbon membrane treated in a similar manner to Example 1 was cut off in a size of 18 cm² and used.

### Example 13

### Preparation of the Polyethylenimine-Coated Porous Carbon Membrane

The porous carbon membrane obtained by the oxidation-treatment of Example 1 was cut off in about 2 cm square and immersed into the ethanol solution containing 0.2 wt % polyethylenimine (hereafter, abbreviated as PEI, average molecular weight 10,000 made by Aldrich), and repeatedly exposed to reduced pressure and ordinary pressure several times, followed by gently shaking at 40° C. for one hour. The membrane was washed with purified water and dried with suction on a Kiriyama's funnel, followed by drying under reduced pressure in a desiccator to give the polyethylenimine-coated porous carbon membrane. By a treatment like this, the porous carbon membrane in which polyethylenimine is introduced onto its surface can be also obtained.

### Example 14

### Immobilization of the Osmium Complex Polymer and PQQ-Dependent Glucose Dehydrogenase (GDH) onto the Polyethylenimine-Coated Porous Carbon Membrane by the Layer-By Layer Stacking Method (Polyacrylic Acid Molecular Weight 25,000)

As the polycation solution and the polyanion solution, were used those having the compositions of Example 9. The molecular weight of polyacrylic acid was 25,000.

The polyethylenimine-coated porous carbon membrane obtained in Example 13 was cut off in a about 2 cm square, and the following operations were carried out.

(1) The membrane is washed with purified water while conducting suction filtration on a Kiriyama's funnel. After confirming that no water remained on the membrane, it was immersed into the polyanion solution in wells of a polystyrene plate with six-wells, and repeatedly exposed to reduced pressure and ordinary pressure in a desiccator to replace the air in the membrane with the immobilization solution. Then, the entire plate is centrifuged at 1500 g for 10 minutes and left for 10 minutes while gently shaking on a plate shaker.

(2) Then, the membrane is collected and washed with purified water while conducting suction filtration on a Kiriyama's funnel. After confirming that no water remains on the membrane, it is immersed into purified water in a glass petri dish (diameter 4 cm), and repeatedly exposed to reduced pressure and ordinary pressure in a desiccator to replace the air in the membrane with the immobilization solution. Then, the membrane is collected and washed with purified water while conducting suction filtration on a Kiriyama's funnel.

(3) After confirming that no water remains on the membrane, it is immersed into the polycation solution in wells of a six-wells plate, and repeatedly exposed to reduced pressure and ordinary pressure in a desiccator to replace the air in the membrane with the immobilization solution. Then, the entire plate is centrifuged at 1500 g for 10 minutes and left for 10 minutes while gently immersing on a plate shaker.

Since, by the above-described operations 1 to 3, a single layer of the layer-by-layer stacking membrane is formed, the repeating number of this operation gives the number of layers. For example, the five-layer stacked membrane is obtained by repeating the operation five times.

Then, the membrane was collected and washed with purified water while conducting suction filtration on a Kiriyama's funnel. After confirming that no water remained on the membrane, it was dried in a vacuum desiccator and stored at −20° C.

### Example 15

### Immobilization of the Osmium Complex Polymer and PQQ-Dependent Glucose Dehydrogenase (GDH) onto the Polyethylenimine-Coated Porous Carbon Membrane by the Layer-By Layer Stacking Method (Polyacrylic Acid Molecular Weight 5,000)

The same operation as Example 14 was carried out except that the polyanion solution used was a solution in which polyacrylic acid (molecular weight 5,000) was dissolved in purified water and adjusted to pH 6 with 1 mold NaOH, followed by diluting with purified water to the final concentration of 1 mg/ml.

### Example 16

### Immobilization of the Osmium Complex Polymer and PQQ-Dependent Glucose Dehydrogenase (GDH) onto the Polyethylenimine-Coated Porous Carbon Membrane by the Layer-By Layer Stacking Method (Polyacrylic Acid Molecular Weight 250,000)

The same operation as Example 14 was carried out except that the polyanion solution used was a solution in which polyacrylic acid (molecular weight 250,000) was dissolved in purified water and adjusted to pH 6 with 1 mol/l NaOH, followed by diluting with purified water to the final concentration of 1 mg/ml.

### Example 17

### Immobilization of the Metal Nanoparticle-Carrying Protein (Ferritin) onto the Polyethylenimine-Coated Porous Carbon Membrane by the Layer-By Layer Stacking Method

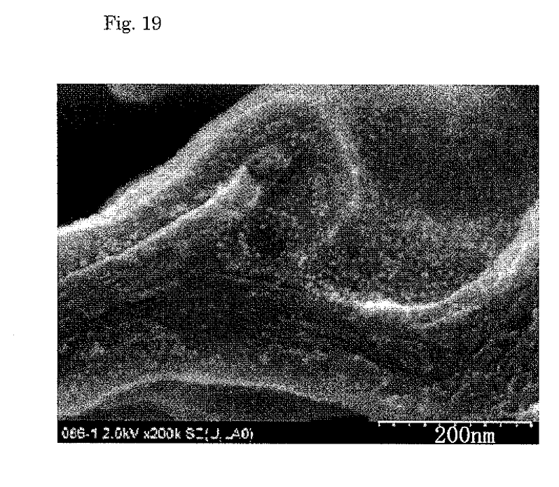

The same operation as Example 14 was carried out except for using those having the composition described in Example 11 as the polycation solution and the polyanion solution. FIG. 18 shows the result of the EPMA analysis conducted for the five-layers stacked membrane likewise Example 3. In comparison with the result (FIG. 17) of Example 11, the distribution of iron element was improved and ferritin was immobilized on the membrane surface of the entire membrane. It is considered that this result is demonstrating the effect of using an organic solvent having a low viscosity in the polycation solution, in comparison with an aqueous solution, as the first treating solution. FIG. 19 also shows the cross-section SEM image. It is observed that the immobilized layers were formed on the pore surface of the carbon membrane, and the ferritin particles were present inside of them.

### Example 18

### Immobilization of the Osmium Complex Polymer and PQQ-Dependent Glucose Dehydrogenase (GDH) onto the Porous Carbon Membrane by the Layer-By Layer Stacking Method; Omission of the Operations with Reduced Pressure and Centrifuge

As the polycation solution and the polyanion solution, were used those having the composition described in Example 9.

The porous carbon membrane obtained by the oxidation treatment of Example 1 was cut off in a about 2 cm square, and the following operations were carried out.

(1) The membrane is immersed into the polycation solution in wells of a polystyrene plate with six-wells, and left for 10 minutes while gently shaking on a plate shaker.

(2) Then, the membrane is collected and washed with purified water, and water attaching on the membrane is brought into contact with a filter paper to remove water.

(3) The membrane is immersed into the polyanion solution in wells of a six-wells plate, and left for 10 minutes while gently shaking on a plate shaker.

Since, by the above-described operations 1 to 3, a single layer of the layer-by-layer stacking membrane is formed, the repeating number of this operation gives the number of layers. For example, the five-layer stacked membrane is obtained by repeating the operation five times.

Then, the membrane was collected and washed with purified water while conducting suction filtration on a Kiriyama's funnel. After confirming that no water remained on the membrane, it was dried in a vacuum desiccator and stored at −20° C.

### Comparative Example 1

### Immobilization of the Osmium Complex Polymer and PQQ-Dependent Glucose Dehydrogenase (GDH) onto Carbon Paper by the Layer-By Layer Stacking Method

The nitric acid-treated carbon paper was obtained by conducting the oxidation treatment of Example 1 using carbon paper (made by Toray: TGP-H-030) instead of the porous carbon membrane. The enzyme and mediator were immobilized on the carbon paper by the same operations as Example 9 except for using this nitric acid-treated carbon paper instead of the porous carbon membrane.

### Referential Example 4

### Three-Dimensional Immobilization of the Osmium Complex Polymer and Glucose Dehydrogenase (GDH) onto the Porous Carbon Membrane

5 mg/ml of glucose dehydrogenase solution 9.6 μl, 5 mg/ml osmium complex polymer solution 2.9 μl synthesized in Referential Example 3, and 1 mg/ml poly(ethyleneglycol)diglycidyl ether (made by Aldrich, average molecular weight 528: hereafter abbreviated as PEGDGE) solution 2.9 μl were applied on the porous carbon membrane (diameter 3 mm) synthesized likewise Referential Example 2, and dried in the air, followed by drying for 16 hours in a desiccator to obtain the enzyme-immobilized membrane, which was stored at −20° C.

### Referential Example 5

### Three-Dimensional Immobilization of Potassium Ferricyanide and Bilirubin Oxidase (BO) onto the Porous Carbon Membrane

5 mg/ml of bilirubin oxidase solution 15 μl, 5 mg/ml of potassium ferricyanide solution 6 μl, 5.0 mg/ml of polyallylamine solution 6 μl, and 1 mg/ml poly(ethyleneglycol)diglycidyl ether (made by Aldrich, average molecular weight 528) solution 6.0 μwere applied on the porous carbon membrane (diameter 3 mm) synthesized likewise Referential Example, and dried in the air, followed by drying for 16 hours in a desiccator to obtain the enzyme-immobilized membrane, which was stored at −20° C.

### Experimental Example 2 for the Sensor

As a electrochemical analyzer, the cell was constructed using the BAS-made model-600A glassy carbon electrode (made by BAS, ID 3 mm) in which the porous carbon membrane of a measurement target was physically adhered on the electrode surface as a working electrode, Ag/AgCl electrode (made by BAS, RE-1B) for a reference electrode, and Pt mesh electrode (made by BAS) for a counter electrode. The measurement was carried out under nitrogen atmosphere at 25° C.

For the electrolyte solution when the immobilized enzyme was Gox, 10 ml of 20 M phosphate buffer solution (pH 7.0) containing 0.1 M NaCl was used. In the case of GDH, 10 ml of 20 mM MOPS buffer solution (pH 7.0) containing 0.1 M NaCl and 2 mM CaCl₂ was used.

The electrolyte solution containing the predetermined concentration of glucose was added to the electrochemical cell, after stirring by a magnetic stirrer for 15 minutes. The cyclic voltammetry (CV) measurement and the chronoamperometry measurement were carried out. The CV measurement was carried out at a potential scanning rate of 1 mV/s. The chronoamperometry measurement was carried out where voltage from 0 V to +0.2 V was applied and the electrical current value was measured after 5 minutes.

(Measurement of Dependence on the Stacking Number)

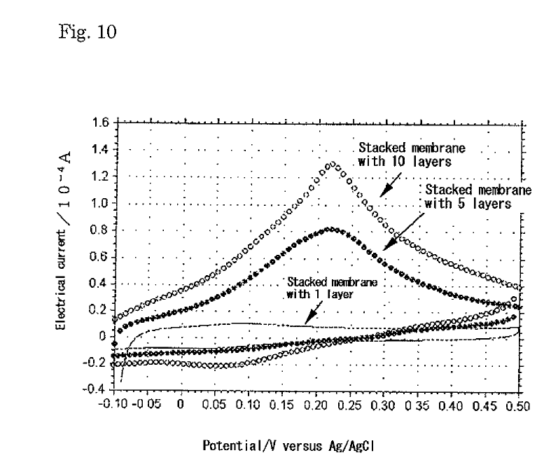

The electrode equipped with the GDH-immobilized membrane with varied numbers of the stacking layers by the layer-by-layer stacking method in Example 9 was prepared. Each electrode was immersed into 100 mM concentration of glucose solution, and the electrical current value was measured by the above-described measuring method. Consequently, as shown in FIG. 10, an increase in the response was observed depending on the stacking number. From this result, it was indicated that the layer-by-layer stacking method increases the amount of the enzyme and metal complex in a useable form.

(Comparison between the porous carbon membrane and the carbon paper)

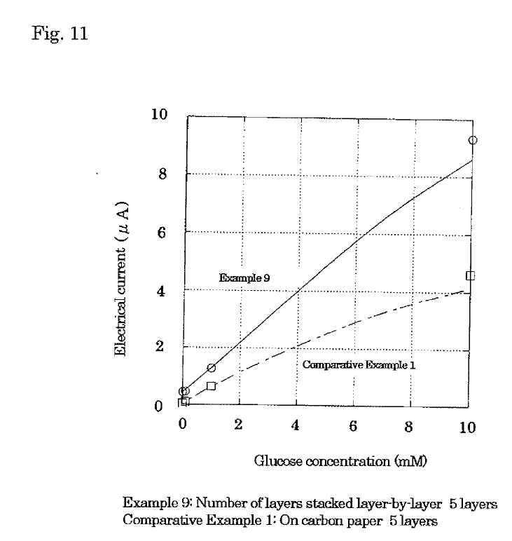

FIG. 11 shows the result by evaluating the dependency on glucose concentration by using the GDH-immobilized porous carbon membrane stacked with five layers in Example 9 and the GDH-immobilized carbon paper stacked with five layers in Comparative Example 1 respectively as the electrode. From this result, it was indicated that the present invention using the porous carbon membrane has superior response.

(Comparison between Treatments and Operations)

The GDH-immobilized membranes with five of the stacking number were produced as in Example 9, Example 14 and Example 18. The electrodes were immersed in 100 mM concentration of glucose solution to evaluate the electrodes by the chronoamperometry measurement.

TABLE 10

| | | Example 14 | Example 18 |
|---|---|---|---|
| | | (after the | (without the centri- |
| | | PEI-coating | fugal treatment under |
| | Example 9 | treatment) | reduced pressure) |
| Electrical | 12.58 | 16.11 | 10.28 |
| current | | | |
| (μA) | | | |

When the enzyme was immobilized by the layer-by-layer stacking method in Example 14, after the porous carbon membrane was oxidation-treated, the membrane was first treated with polyethylenimine dissolved in an organic solvent and the enzyme was subsequently immobilized by the layer-by layer method. Improvement was further observed in the chronoamperometry response compared with Example 9 without the PEI-coating treatment. Example 18 was an example for the attempt to immobilize the enzyme without the centrifugal treatment or reduced pressure. The chronoamperometry response was increased in Example 9, which comprises these treatments. Therefore, it was obviously effective as the immobilization method not only to simply immerse the porous carbon membrane into the solution containing the immobilization target while immobilizing the biological molecule, but also to treat the entire system under reduced pressure or centrifuge during immersing.

(Comparison between molecular weights of the polyanion)

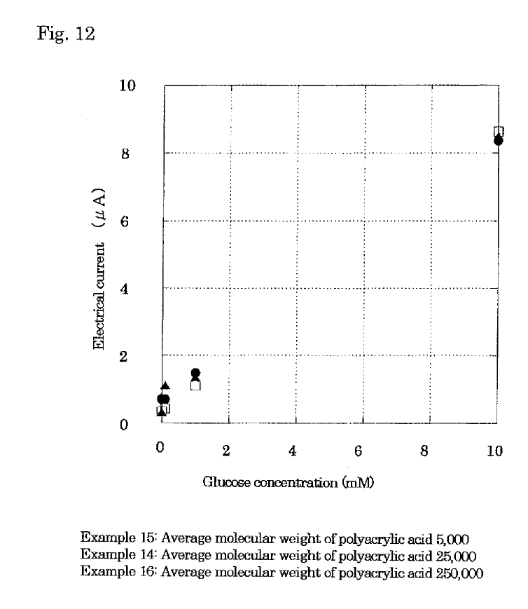

FIG. 12 shows the result of the dependency on glucose concentration by using the GDH-immobilized porous carbon membrane stacked with five layers produced in Example 14, Example 15, and Example 16 as respective electrodes. The average molecular weights of the polyacrylic acids used were 25,000 in Example 14, 5,000 in Example 15, and 250,000 in Example 16. Excellent responses for the glucose concentration were shown in every example.

### Experimental Example 3 for the Sensor: FIA

Next, is explained a structural example of the sensor used for the Flow Injection Analysis (FIA) conducting the measurement while flowing the measurement target through.

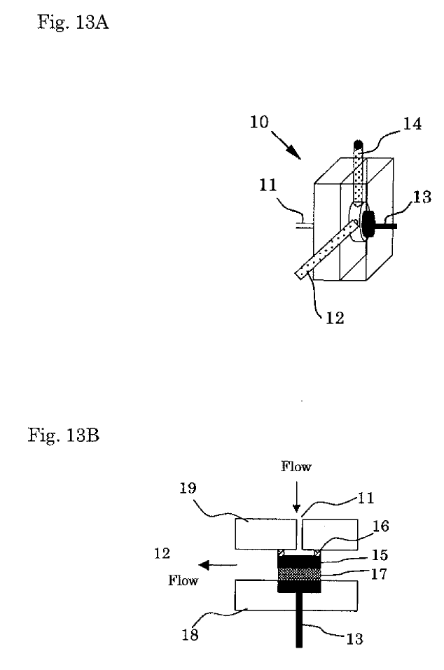

Using the circle with a diameter of 3 mm and a film thickness of 80 μm of the porous carbon membrane, the enzyme-immobilized porous carbon membrane was obtained, in which the osmium complex polymer and PQQ-dependent glucose dehydrogenase (GDH) were immobilized in accordance with Example 9. Using the obtained carbon membrane and utilizing the radial flow cell made by BAS, an instrument indicated in FIG. 13A and FIG. 13B was made. As shown in FIG. 13A, this sensor 10 is equipped with the inlet 11 of the measurement solution, the outlet 12 (also serving as the auxiliary electrode) of the measurement solution, the working electrode 13 and the reference electrode 14. In the sensor as shown in FIG. 13B, the porous carbon paper 17 and the enzyme-immobilized porous carbon membrane 15 are placed on the working electrode 13 inside the lower supporting frame 18, and sandwiched between the lower supporting frame 18 and the upper supporting frame 19 through the Teflon ring 16. The measurement solution is injected from the inlet 11 of the measurement solution to fill up the room surrounded by the Teflon ring 16 and contact the membrane face of the enzyme-immobilized porous carbon membrane 15, and is filled into the membrane. Although a part of the measurement solution laterally seeps out from the carbon membrane 15, the most part flows in the direction of the membrane thickness, flows through the porous carbon membrane 17 and laterally flows out from the carbon paper, and they are collected to flow out from the outlet 12 of the measurement solution. For the porous carbon paper, those having the higher vacancy ratio than that of the enzyme-immobilized porous carbon membrane 15 are used. Since the carbon paper is also electrically conductive, the enzyme-immobilized porous carbon membrane 15 is electrically connected to the working electrode 13 and serves as a functional portion of the working electrode.

By using the instrument like this, the electrolyte solution previously-described in <Experimental Example 2 for the sensor> was flowed at a flow rate of 10 μl/min as a mobile phase. The sample containing the predetermined concentration of glucose dissolved in the mobile phase was injected in 10 μl, and the chronoamperometry measurement was initiated at the same time as the injection. FIG. 14 shows the result by plotting the peak electrical current value caused by the reaction with glucose against the glucose concentration. From this result, high correlation between the glucose concentration and the peak electrical current value was observed. Since the biological molecule-immobilized carbon membrane of the present invention also enables the immobilization of the mediator and has air permeability and fluid permeability, it is also suitable for a flow-type sensor.

### Experimental Example 1 for the Bio-Fuel Cell

The biological molecule-immobilized carbon membrane produced in the example and referential example was physically adhered on the electrode surface of the glassy carbon electrode (made by BAS, ID 3 mm) to produce an electrode. The measurement was carried out under oxygen atmosphere at 25° C. For the electrolyte solution, 10 ml of 20 mM MOPS buffer solution (pH 7.0) containing 0.1 M glucose, 0.1 M NaCl and 2 mM CaCl₂ was used. While changing a resistance load between both electrodes from 2 M to 100Ω, the electrical current and voltage were measured. Table 11 shows that result.

TABLE 11

| | Maximum output |
|---|---|
| Electrode constitution 1: | 160 μW/cm² |
| Layer-by-layer stacking method | |
| immobilization | |
| Electrode constitution 2: | 60 μW/cm² |
| Three-dimensional gel immobilization | |

Here, the constitutions of the anode and cathode are as follows.

Electrode Constitution 1

Anode: the enzyme-immobilized porous carbon membrane (GDH and the osmium complex polymer were immobilized) having five stacked layers in Example 9.

Cathode: the enzyme-immobilized porous carbon membrane (BO and the potassium ferricyanide were immobilized) having five stacked layers in Example 10.

Electrode Constitution 2

Anode: the carbon membrane (GDH and the osmium complex polymer were immobilized) prepared by the three-dimensional immobilization of Referential Example 4.

Cathode: the carbon membrane (BO and the potassium ferricyanide were immobilized) prepared by the three-dimensional immobilization of Referential Example 5.

Although the both electrode constitutions gave the output power, higher maximum output was obtained in the electrodes having stacked layers obtained by the layer-by-layer stacking method.

### Experimental Example 2 for the Bio-Fuel Cell: The Chip-Type Bio-Fuel Cell

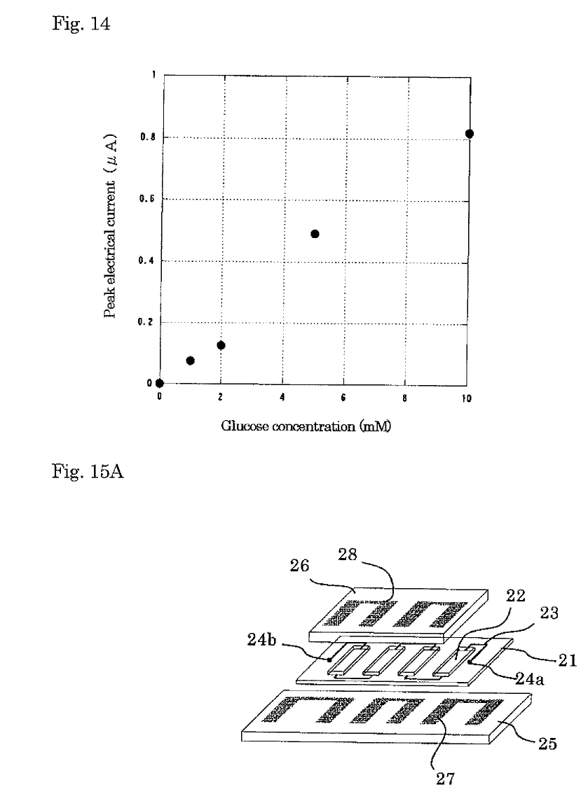

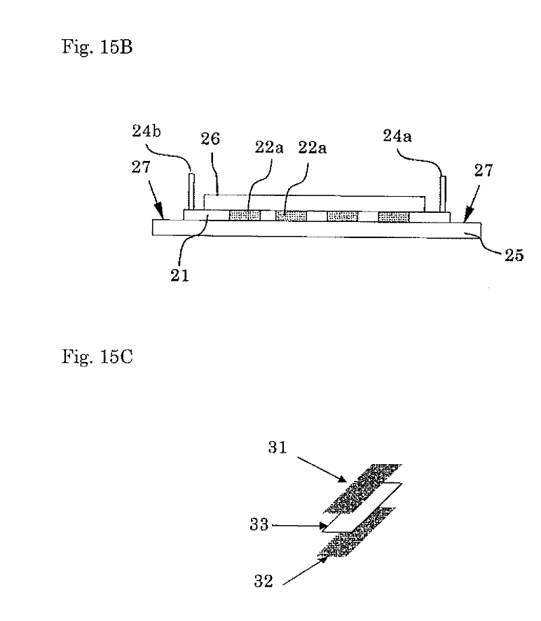

FIG. 15A to FIG. 15C show an example of the chip-type bio-fuel cell. The through-holes 22 for the cell with a size of 6 mm and 12 mm, and the flow path 23 to interconnect the adjacent through-holes 22 were formed on the silicone rubber (polydimethylsiloxane) plate 21. As shown in FIG. 15A, the lower glass substrate 25 on which the platinum film electrode 27 was formed was prepared, on which the processed silicone rubber was adhered so that the through-holes 22 for the cell fits four electrodes for the cell at the center. The porous carbon membrane, the membrane filter, and the porous carbon membrane were stacked by this order and placed in the through-holes 22 for the cell. The upper glass plate 26 on which the platinum film electrode 27 was formed was prepared, and four electrodes for the cell were positioned at the through-holes 22 of the silicone rubber 21, which was sandwiched by the glass substrates 25 and 26 from upper and lower sides.

FIG. 15B is the cross-sectional view of this chip-type bio-fuel cell. Sandwiching the glass plates from upper and lower sides formed a structure having the connection of four cells 22a through the flow path 23. At the both terminals of the flow path 23, the glucose injection inlet 24a and the glucose outlet 24b were also assembled. The terminal part of the electrode 27 on the lower glass substrate 25 was designed so as to be exposed after assembled.

FIG. 15C is a figure showing the cell constitution, in which the membrane filter 33 is sandwiched by the porous carbon membrane 31 for the cathode and the porous carbon membrane 32 for the anode. These single-cell structures are placed at the through-holes 22 upside down between the adjacent cells to construct the cell in which four single-cell structures are connected serially.

Here, the enzyme-immobilized porous carbon membranes with a size of 5 mm×10 mm×0.1 mm were prepared in accordance with Example 10 and Example 9 for the cathode and for the anode, respectively.

20 mM MOPS buffer solution (pH 7.0) containing 0.1 M glucose, 0.1 M NaCl and 2 mM CaCl₂ was bubbled with oxygen in advance, and introduced into the flow path, and the electrical current and voltage were measured while changing the resistance load from 2 M to 100Ω. As a result, the fuel cell showed the output power of 0.75 V as open voltage and 48 μW as maximum output power.

### Experimental Example 3 for the Bio-Fuel Cell: The Polymeric Electrolyte Membrane-Type Bio-Fuel Cell

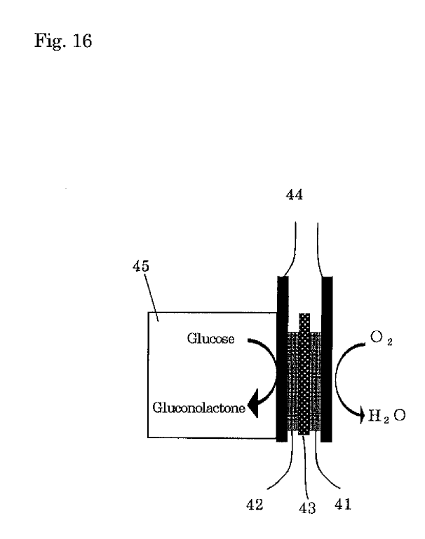

Serpentine Flow (C05-01SP-REF: electrode area 5 cm²) made by ElectroChem was used for constructing the polymeric electrolyte membrane-type fuel cell. The porous carbon membrane with an area of 5 cm² prepared in Example 8 and Example 9 was used for the anode, an electrode (1 mg/cm² Pt(20 wt % Pt/XC-72) made by ElectroChem was used for the cathode, and an acid-treated Nafion 112 was used for the polymeric electrolyte membrane. In the production of the cell, the acid-treated Nafion membrane and the cathode were hot-pressed (130° C., one minute), and then the enzyme-immobilized carbon membrane was pressed at room temperature for 2 minutes. As schematically shown in FIG. 16, the cell has a structure in which the proton conductor (Nafion 112) 43 is sandwiched by the positive electrode 41 and the negative electrode 42, and the power collectors 44 are equipped with outsides of each electrode.

For the electrolyte solution (fuel solution) 45 when the immobilized enzyme was Gox, 100 mM phosphate buffer solution (pH 7.0) containing 0.1 M glucose were used. In the case of GDH, 20 mM MOPS buffer solution (pH 7.0) containing 0.1 M glucose, 0.1 M NaCl, and 2 mM CaCl₂ was used. While changing the resistance load between both electrodes from 2 M to 100Ω, the electrical current and voltage were measured. During the power generation, pure oxygen 20 ml/min was supplied to the cathode electrode, and the electrolyte solution was supplied to the anode electrode at 1 ml/min. Table 12 shows the result.

TABLE 12

| Immobilized enzyme | Maximum output |
|---|---|
| Gox (Example 8) | 18 μW/cm² |
| GDH (Example 9) | 7 μW/cm² |

The device examples of the sensors and bio-fuel cells shown in the above examples are intended to demonstrate that the biological molecule-immobilized carbon membrane of the present invention is applicable for the sensors and bio-fuel cells. It is obvious to a person skilled in the art that devices with various structures are possible by arranging the electrodes properly.

1. A biological molecule-immobilized carbon membrane, wherein the biological molecule is immobilized onto a porous carbon membrane having fluid-permeable three-dimensional cancellous pores.

2. A biological molecule-immobilized carbon membrane according to claim 1, wherein the porous carbon membrane has an air permeability of 10 to 2,000 sec/100 cc, and a specific surface area of 1 to 1,000 m²/g.

3. A biological molecule-immobilized carbon membrane according to claim 1, wherein an electrostatic interaction of the porous carbon membrane surface and the biological molecule causes the immobilization of the biological molecule.

4. A biological molecule-immobilized carbon membrane according to claim 3, wherein an anion group is introduced onto the surface of the porous carbon membrane by an oxidation treatment, and the electrostatic interaction of this surface anion group and a positive charge in the biological molecule causes the immobilization of the biological molecule.

5. A biological molecule-immobilized carbon membrane according to claim 3, wherein a compound having a cation group is introduced onto the surface of the porous carbon membrane after an oxidation treatment, and the electrostatic interaction of this surface cation group and a negative charge in the biological molecule causes the immobilization of the biological molecule.

6. A biological molecule-immobilized carbon membrane according to claim 1, wherein a covalent bond between a surface of the porous carbon membrane and the biological molecule causes the immobilization of the biological molecule.

7. A biological molecule-immobilized carbon membrane according to claim 1, wherein a physical interaction of the porous carbon membrane surface and the biological molecule causes the immobilization of the biological molecule.

8. A biological molecule-immobilized carbon membrane according to claim 1, comprising a first polymeric electrolyte having a charge opposite to a charge of the biological molecule, and forming an ion complex by an electrostatic interaction with the biological molecule.

9. A biological molecule-immobilized carbon membrane according to claim 8, wherein the biological molecule and the first polymeric electrolyte are alternately stacked to form the ion complex.

10. A biological molecule-immobilized carbon membrane according to claim 8, further comprising a second polymeric electrolyte having the same charge as the biological molecule, and forming the ion complex with the first polymeric electrolyte in a manner where the biological molecule and the second polymeric electrolyte are mixed.

11. A biological molecule-immobilized carbon membrane according to claim 8, wherein the anion group is introduced onto the surface of the porous carbon membrane before introducing the biological molecule.

12. A biological molecule-immobilized carbon membrane according to claim 8, wherein the anion group is introduced onto the surface of the porous carbon membrane before introducing the biological molecule, followed by a treatment with an organic solvent solution of the compound having the cation group.

13. A biological molecule-immobilized carbon membrane according to claim 1, wherein the biological molecule is a protein or a nucleotide.

14. A sensor comprising the biological molecule-immobilized carbon membrane according to claim 1 as an electrode.

15. A bio-fuel cell comprising the biological molecule-immobilized carbon membrane according to claim 1 as an electrode.

16. A process for producing a biological molecule-immobilized carbon membrane, comprising the steps of:
providing a porous carbon membrane having a three-dimensional cancellous pore, an air permeability from 10 to 2,000 sec/100 cc, and a specific surface area from 1 to 1,000 m²/g;
oxidation-treating the porous carbon membrane; and
immersing the porous carbon membrane after oxidation treatment in a solution containing the biological molecule to immobilize the biological molecule onto the porous carbon membrane.

17. A process for producing a biological molecule-immobilized carbon membrane, comprising the steps of:
providing a porous carbon membrane having a three-dimensional cancellous pore, an air permeability from 10 to 2,000 sec/100 cc, and a specific surface area from 1 to 1,000 m²/g;
oxidation-treating the porous carbon membrane;
introducing a cation group onto a surface of the porous carbon membrane after oxidation treatment; and
immersing the porous carbon membrane after the cation group has been introduced in a solution containing the biological molecule to immobilize the biological molecule onto the porous carbon membrane.

18. A process for producing a biological molecule-immobilized carbon membrane, comprising the steps of:
providing a porous carbon membrane having a three-dimensional cancellous pore, an air permeability from 10 to 2,000 sec/100 cc, and a specific surface area from 1 to 1,000 m²/g;
oxidation-treating the porous carbon membrane; and
immobilizing the biological molecule onto the porous carbon membrane through a covalent bond.

19. A process for producing a biological molecule-immobilized carbon membrane, comprising the steps of:
providing a porous carbon membrane having a three-dimensional cancellous pore, an air permeability from 10 to 2,000 sec/100 cc, and a specific surface area from 1 to 1,000 m²/g; and
bringing a mixture containing the biological molecule and a crosslinkable compound into contact with the porous carbon membrane to immobilize the biological molecule onto the porous carbon membrane.

20. A functional carbon membrane, wherein is oxidized a surface of a porous carbon membrane having a fluid-permeable three-dimensional cancellous pore, followed by introducing a compound having a cation group.

21. A functional carbon membrane according to claim 20, wherein the porous carbon membrane has an air permeability of 10 to 2,000 sec/100 cc, and a specific surface area of 1 to 1,000 m²/g.

22. A biological molecule-immobilized carbon membrane according to claim 13, wherein the biological molecule is selected from the group consisting of glucose dehydrogenase, glucose oxidase, bilirubin oxidase, diaphorase, alcohol dehydrogenase, avidin and biotin.

23. A process for producing a biological molecule-immobilized carbon membrane, comprising the steps of:
providing a porous carbon membrane having three-dimensional cancellous pores, an air permeability from 10 to 2,000 sec/100 cc, and a specific surface area from 1 to 1,000 m²/g;
providing a solution (a) and a solution (b), wherein the solution (a) contains one or more polymeric electrolytes with a positive charge and the solution (b) contains one or more polymeric electrolytes with a negative charge, and wherein at least one of the polymeric electrolyte with the positive charge and the polymeric electrolyte with the negative charge is the biological molecule; and
alternately stacking each membrane at least once by alternately conducting the sub-steps of:
(a) immersing the porous carbon membrane in the solution (a) and
(b) immersing the porous carbon membrane in the solution (b).

24. A production process according to claim 23, further comprising the step of oxidation-treating the porous carbon membrane before the alternate stacking, and
wherein the sub-step (a) is conducted first during the alternate stacking.

25. A production process according to claim 23,
wherein the production process comprising, prior to the step of alternate stacking, the steps of oxidation-treating the porous carbon membrane, and introducing a cation group onto a surface of the porous carbon membrane after oxidation treatment; and
wherein the step of alternate stacking starts from the sub-step (b).

26. A production process according to claim 23, wherein either the solution (a) or the solution (b) contains the biological molecule, and the other contains a mediator.

27. A production process according to claim 23, wherein either the solution (a) or the solution (b) contains both the biological molecule and the mediator.
